(19) United States
(12) Patent Application Publication
(10) Pub. No.: US 2021/0101929 A1
(43) Pub. Date: Apr. 8, 2021

(54) METHOD FOR STABILIZING PROTEIN COMPRISING FORMULATIONS BY USING A MEGLUMINE SALT

(71) Applicant: MERCK PATENT GMBH, DARMSTADT (DE)

(72) Inventors: Christoph KORPUS, Frankfurt am Main (DE); Raphael Johannes GUEBELI, Darmstadt (DE)

(73) Assignee: MERCK PATENT GMBH, DARMSTADT (DE)

(21) Appl. No.: 17/048,514

(22) PCT Filed: Apr. 16, 2019
(86) PCT No.: PCT/EP2019/059771

§ 371 Date: Oct. 16, 2020

(30) Foreign Application Priority Data
Apr. 16, 2018 (EP) 18167607.3

(51) Int. Cl. C07K 1/02; C07K 16/00; A61K 47/18; A61K 47/26

(52) U.S. Cl. CPC C07K 1/02; C07K 16/00; A61K 9/16; A61K 47/26; C07K 2317/94; A61K 47/183

(57) ABSTRACT

The present invention relates to a method for stabilizing protein or peptide comprising formulations, which includes the step of adding selected meglumine salts to protein solutions, especially to solutions of pharmaceutical active proteins. But the present invention also relates to the stabilized composition comprising proteins or peptides and selected meglumine salts. Another objective of the present invention is to provide pharmaceutical compositions comprising antibody molecules stabilized by selected meglumine salts and methods for producing corresponding stabilized pharmaceutical compositions, and kit comprising these compositions.

The present invention relates to a method for stabilizing protein or peptide comprising formulations, which includes the step of adding selected meglumine salts to protein solutions, especially to solutions of pharmaceutical active proteins. But the present invention also relates to the stabilized composition comprising proteins or peptides and selected meglumine salts. Another objective of the present invention is to provide pharmaceutical compositions comprising antibody molecules stabilized by selected meglumine salts and methods for producing corresponding stabilized pharmaceutical compositions, and kit comprising these compositions.

### STATE OF THE ART

Protein stability is a major challenge during the development of protein therapeutics (Wang, W.; Int J Pharm, 185(2) (1999) 129-88; “Instability, stabilization, and formulation of liquid protein pharmaceuticals”) and needs to remain under tight control to assure efficacy of the protein drug and to ensure patient safety. The importance of stability during the development of protein therapeutics is also recognized by regulatory authorities. Forced degradation studies according to ICH Q5C or the identification and mitigation of protein particles are crucial stability-indicating measures during development (Hawe, A.; Wiggenhorn, M.;van de Weert, M.; Garbe, J. H.; Mahler, H. C. and Jiskoot, W.; J Pharm Sci, 101: (2012) 895-913; “Forced degradation of therapeutic proteins”).

The most common strategy in the biopharmaceutical industry to increase stability of proteins relies on the addition of stabilizing excipients to protein solutions (Improvement of the stability of a protein by changing the peptide sequence is not addressed in this invention). Excipients are traditionally used to stabilize the finally formulated product, either in liquid or lyophilized state. However, it is worth mentioning that the similar concept of stabilization can also be applied to the whole manufacturing process, e.g. during cell culture or the downstream purification process.

To date, a scientist skilled in the art can choose from a handful of excipients for the stabilization of proteins. One family of stabilizers consists of sugars and polyols such as sucrose, trehalose, mannitol and sorbitol. They stabilize the protein by acting as excluded solvents (Arakawa, T.; Timasheff, S. N.; Biophysical Journal, 47: (1985) 411-14; “The stabilization of proteins by osmolytes”). On the other hand, stabilization can be also achieved by modifying the protein's charge interactions using charged excipients such as NaCl and Arginine.

Recently, meglumine (N-Methyl-D-glucamin) has been proposed as a potential protein-stabilizing excipient (Igawa, T. C. S. K. K.; Kameoka, D. C. S. K. K.; U.S. Pat. No. 8,945,543 B2 (2008); “Stabilizer for protein preparation comprising meglumine and use thereof”; and Manning, M.; Murphy, B.; US 2013/108643 A1 (2013); “Etanercept Formulations Stabilized with Meglumine”).

In this context it has been assumed that meglumine would be capable of reducing the aggregation of proteins, while it can take over the effects of a containing solvent and of a charge modifier with the effect that the latter were not anymore needed in the protein formulation.

However, more detailed analyzes of the formulations, which are disclosed in this document, show that sufficient stabilization of the proteins used in more recent formulations can not be achieved in this way.

### Object of the Present Invention

For a significant number of proteins in development, especially novel protein formats such as fusion proteins, attempts for stabilization using commonly known excipients still fail. This is why there is still a need in the biopharmaceutical industry to provide suitable excipients showing improved stabilizing properties, especially for these new protein formats.

### Subject-Matter of the Invention

The subject of the present invention is a method of stabilizing of a liquid protein or peptide formulation or for suppressing protein aggregation in said formulation by treatment of the peptide- or protein-containing solution with a combination of meglumine and a physiologically well-tolerated organic counterion in effective concentrations to stabilize the protein or peptide molecules contained therein. In a selected embodiment of the invention the method for the stabilization of a liquid protein or peptide formulation or for suppressing protein aggregation is carried out by
(a) providing a first solution comprising protein or peptide molecules; and
(b) providing a second solution comprising meglumine in combination with a selected, physiologically well-tolerated organic counterion in a suitable formulation,
(c) adding a sufficient amount of the second solution to the first solution, and
   thereby setting in the resulting mixture a meglumine-counterion concentration, which is effective for stabilization the comprising protein or peptide molecules.

Furthermore, the present invention encompasses the further embodiments of this method as claimed by claims 3 to 18 and the pharmaceutical protein or peptide formulations of claims 19 and 20 produced and stabilized by this method. Another object of the present invention is a kit containing the protein formulations according to the invention of claims 21 to 24.

### DETAILED DESCRIPTION OF THE INVENTION

Although there are a variety of studies on suitable pharmaceutical formulations for the effective application of proteins, these agents are still preferably administered subcutaneously in solution. Therefore, the stabilization of proteins is an essential task for the formulator, because in solutions the preferred interaction of the protein is usually with either water or the added excipients. In the presence of a stabilizing excipient, the protein preferably is “surrounded” by water molecules (preferential hydration), since the excipient from the environment of the protein is usually excluded (preferential exclusion). This represents a thermodynamically favorable state for native proteins, so that the physical denaturation is prevented [Stabenau, Anke; in “Trocknung und Stabilisierung von Proteinen mittels Warmlufttrocknung und Applikation von Mikrotropfen”, Dissertation München 2003].

In literature, there are various examples of stabilizers that prevent aggregation and denaturation of the protein molecules through steric hindrance.

Other additives, in turn, cause an increase in the melting temperature (Tₘ) of proteins or decrease the adsorption to surfaces of other proteins, which leads to an attachment on the surface of the protein molecules, which then can lead to changes in the protein itself and to a loss of its activity. In order to prevent this, attempts have been made to work with small amounts of surface-active compounds, such as polysorbates. But depending on the chemical structure, these additives used to stabilize the proteins also may have undesirable disadvantages, i. e. as said polysorbates, which may be subject to autoxidation and thereby may lead to the release of hydroperoxides, side-chain cleavage and eventually formation of short chain acids such as formic acid and all of which can influence the stability of a biopharmaceutical composition.

As can be seen from the above, various substances are described in literature as suitable for the stabilization of protein formulations. These include sugars such as sucrose or trehalose, polyalcohols such as mannitol or sorbitol, amino acids such as glycine, arginine, leucine or proline, surfactants like polysorbates, and other stabilizers like human serum albumin. However, most of these substances as such show more or less strong effects, which must be balanced by the addition of other additives to retain the activity of the proteins and others do not show sufficient stabilizing effects.

In addition, the activity of each protein formulation depends on the adjustment of the correct pH and the choice of the optimal buffer system.

In terms of the correct pH, most proteins differ, though for almost all, their stability can only be maintained if the pH is kept in a very narrow range. Outside this range, charged groups are formed, electrostatic repulsions, and false salt bridges, resulting in protein denaturation.

However, in addition to the consideration of all chemical and physical instabilities, the applicability of the protein formulation must be kept in mind, as not every pH value is tolerated by the patient. Therefore, the solutions should be as close as possible to the physiological pH of 7.4. While some deviations can be accepted by intravenous administration because of the rapid dilution, but solutions to be administered intramuscular or subcutaneous should be isohydric. In most cases, the pH value present in the product represents a compromise between compatibility and storage stability. In addition, the fundamental question of the physicochemical stability of the proteins persists during storage until administration.

With this background knowledge it has now been looked for a suitable possibility to stabilize pharmaceutically active protein solutions, which itself does not arise any unwanted new side effects.

In this context, meglumine has proved to be a very promising substance in our experiments. Meglumine is already an FDA approved excipient for use in pharmaceutical formulations and which is being used in various X-ray contrast formulations in cancer therapy, and it is also used as part of APIs, which are approved by several regulatory agencies (e.g. small-molecule parenterals) and it has a positive safety track record.

Meglumine can be applied in different administration routes (e.g. oral, intravenous). As a functional excipient where it acts as a counterion it may help to enhance API stability and solubility in formulations However, apart from published data in patents and scientific journals, meglumine has not yet been successfully applied for the stabilization of proteins in the manufacturing or formulation, neither in medicines being approved nor in clinical trials.

Surprisingly, the stabilizing effect of meglumine on protein formulations can be significantly improved, if it is combined with a suitable charged counter ion. Corresponding experiments have shown in this context that the molar ratio of the meglumine and the counterion to one another contained in the formulations is essential for the stabilizing effect, although depending on the overall composition, the optimum ratio may vary. But in particular, when selecting particular conditions, the best stabilization results may be received, if meglumine and the appropriate counterion are added in an equimolar ratio to the formulation. Under these conditions, to stabilize the protein formulation, the corresponding meglumine salt (“meglumine derivative”) may be added directly, preferably in solution.

As such, the protein formulations of the invention may have pH values in the range of pH 5 to 8. As already said, however, it is desirable to provide such protein formulations with a pH value which is optimally adjusted.

Advantageously, by applying a formulation of an equimolar mixture of Meglumine and a counterion in combination with a protein solution it is possible to use pH ranges much closer to the desired level of pH=7.4 than with the use of meglumine and sucrose alone. Therefore, compositions according to the present invention after addition of the meglumine and the counterion preferably have a pH in a range from 7.2 to 7.6, most preferably of 7.4, which is optionally adjusted by the addition of a sufficient amount of a physiologically acceptable add.

Herein “meglumine” refers to the compound represented by the formula 1-Deoxy-1-methylamino-D-glucitol, which is also known as N-methyl-D-glucamine, and compounds represented by the following formula

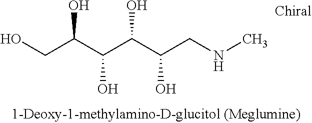

Surprisingly most effective meglumine salts, which show unexpectedly good stabilization effects for pharmaceutically usable protein solutions, are especially glutamates and aspartates of meglumine.

L-glutamic acid is a non-essential, proteinogenic amino acid with an acidic, hydrophilic carboxyl group-bearing side chain. The α-amino acid glutamate or the corresponding α-keto acid α-ketoglutarate plays a prominent role in the metabolism as a nitrogen collection and distribution site.

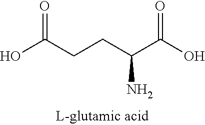

In turn. L-aspartate (L-aspartic acid) is a non-essential, proteinogenic amino acid having a hydrophilic, acidic carboxyl group in the side chain. The amino acid is formed from oxalacetate by adopting a nitrogen group of glutamate. Aspartate is u.a. needed for purine, pyrimidine and urea synthesis.

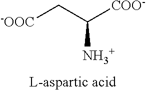

Advantageously, it is found that especially these two counterions for meglumine are compatible in the formulations and since they are amino acids that play an important role in metabolism, which are commonly found in body fluids such as blood, it is not expected that corresponding protein solutions will result in unexpected side reactions when administered subcutaneously.

Now, to stabilize the finally formulated protein product, either in liquid or lyophilized state, means first of all, that in the formulation an irreversible aggregation of the proteins in the solution is avoided as completely as possible. Moreover, it is desirable that this type of stabilization should be applicable throughout the process of preparing the pharmaceutical protein formulations from the moment of protein isolation to completion. Moreover, it is desirable, when thinking of the stabilization of proteins, that in addition to avoiding aggregation, the structural conformation of the proteins is maintained and stabilized.

In order to test these two properties and to study the stabilizing influence thereon by chosen additives, various monoclonal IgG1 antibodies (mAbA and mAbB), as well as a fusion protein (fusionA) were examined in diluted solutions. For this, diluted protein solutions were used at a concentration in the range of 1 mg/ml to 500 mg/ml or higher, which were adjusted to a pH 5 with a phosphate citrate buffer (McIlvaine-buffer). However, it is also possible, under suitable conditions and if necessary, to use solutions in which the concentration is higher than 500 mM and is up to 1.5 M. Preferably the experiments are carried out using protein solutions at a concentration in the range of 1 mg/ml to 50 mg/ml. These solutions were now deliberately mixed with fixed amounts of meglumine and corresponding counterions like glutamate, aspartate and others [meglumine-glutamate (Meg-Glu) and meglumine-aspartate (Meg-Asp)] to test the stabilizing potential.

As a measuring method for demonstrating the improvement of the stabilization, the nanoDSF measurement was selected, which is a modified differential scanning fluorimetry method to determine protein stability employing intrinsic tryptophan or tyrosin fluorescence.

Protein stability is typically addressed by thermal or chemical unfolding experiments. In thermal unfolding experiments, a linear temperature ramp is applied to unfold proteins, whereas chemical unfolding experiments use chemical denaturants in increasing concentrations. The thermal stability of a protein is typically described by the ‘melting temperature’ or ‘Tₘ’, at which 50% of the protein population is unfolded, corresponding to the midpoint of the transition from folded to unfolded. The nanoDSF measurement uses tryptophan or tyrosin fluorescence to monitor protein unfolding. Both the fluorescence intensity and the fluorescence maximum strongly depends on the close surroundings of the tryptophan. Therefore, the ratio of the fluorescence intensities at 350 nm and 330 nm is suitable to detect any changes in protein structure, for example due to protein unfolding.

In summary, the conformational stability is assessed in form of the melting temperature of the protein using differential scanning fluorimetry, wherein the melting temperature (Tₘ) describes at which temperature 50% of the protein is denaturized. Hence an increase in Tₘ is an indicator for an improved protein stability (Menzen, T., and Friess, W. J Pharm Sci, 102: (2013) 415-28; “High-throughput melting-temperature analysis of a monoclonal antibody by differential scanning fluorimetry in the presence of surfactants”).

The results of the experiments clearly show that the protein stabilizing effect of meglumine can be considerably improved if the protein solutions are mixed not only with meglumine but additionally with approximately equimolar amounts of a physiologically tolerated amino acids as charged counterions for meglumine or with other suitable counterions well tolerated by humans.

According to the present invention suitable counterions are those pharmaceutically acceptable organic compounds, which have at least one carboxylic acid group and at least one amino group, but no aromatic groups in the molecule. Particularly good stabilization results are achieved with corresponding dicarboxylic acids as counterions. In this connection, the abovementioned counterions aspartate and glutamate have to be mentioned. But also pharmaceutically acceptable charged compounds are suitable for stabilization, which have at least one carboxylic acid group, at least one amino group and at least one OH group and which can thus act as counterions for meglumine. However, counterions have also been proven to be very suitable, which have no amino group but at least one carboxylic acid group and at least two or more OH groups which, under suitable conditions, have a stabilizing effect on the protein or peptide contained. Counterions of this group do not have any aromatic groups in the molecule. Representative of counterions of this group is for example lactobionate.

Depending on the chemical and physical properties of the compound used as the counterion for meglumine, it may be necessary to add higher amounts of the counterion-acting compound. In some cases, it may therefore be necessary for the counterion compound to be added in excess to the formulation, and thus up to a molar ratio of meglumine to the counterion of 1:2. The optimum molar amount of counterion to be added may accordingly be in a molar ratio of meglumine to counterion between 1:1 to 1:2.

The improved stabilizing effect occurs in particular for protein solutions in which aspartate or glutamate is used as counterion, as can be shown by examples 1A -10. For all model molecules an improved stabilizing effect can be demonstrated here.

In particular, it was found that meglumine-glutamate performed best with an increase in Tₘ of around 3° C. in comparison to solutions comprising meglumine alone. This can be seen very clearly, in Example 1C, in which meglumine glutamate [Meg-Glu] has been mixed in a concentration of up to 500 mM with a solution of a fusion protein (fusionA).

Overall, the carried out experiments show that the addition of meglumine and of a suitable counterion in equimolar amounts can generally stabilize protein solutions, both in terms of undesirable aggregation and in terms of the structural conformation of the protein molecules. However, depending on the counterion used, the amount of counter ion to be used must be adjusted and may require twice the amount. In particular, this applies not only for solutions of monoclonal antibodies but also for solutions of new protein formats, such as of fusion proteins. In addition, it is found that equimolar mixtures of meglumine and of a suitable counter ion increase values of Tₘ even more than the currently most often used protein stabilizing additive, the disaccharide, sucrose.

To assess the stability of proteins in solution, the colloidal stability is often brought into play in connection with aggregation. In this context, the stabilizing potential of the equimolar mixtures of meglumine and the counter ions as named above on the colloidal stability of mAbA and mAbB is also analyzed (Examples 1 D-E).

Both colloidal and conformational stability are assumed to be important in the aggregation of proteins. To successfully stabilize protein against aggregation, solution conditions need to be chosen to not only stabilize the protein native conformation, but also to stabilize the protein against intermolecular attractive forces.

The resistance to aggregation due to native protein-protein interactions in solution is often referred to as the “colloidal stability” of a protein. Today a number of experimental methods are available to determine this stability. Static light-scattering [SLS] arguably provides the most accessible and most developed method for measuring protein-protein interactions in solution and requires only the protein concentration-dependent light-scattering intensity from the protein of interest in the solution of interest.

In general, the SLS measurement at 266 nm is used as an indicator for “colloidal stability”, reporting the onset of aggregation temperature (T_{agg}), which can be defined as the temperature at which the measured scatter reaches a threshold that is approximately 10% of its maximum value.

The changes in the SLS signal represents changes in the weight average molecular mass observed due to protein aggregation. The conformational stability is assessed by measuring the temperature of the on-set of melting, namely the mid-point temperature of the first unfolding transition, Tₘ₁, monitored by an intrinsic fluorescence intensity ratio (350/330 nm) which is sensitive to the tryptophan exposure as protein unfolds (Avacta, 2013b; “Predicting Monoclonal Antibody Stability in Different Formulations Using Optim 2”. Application Note. Avacta Analytical, UK.).

The onset temperature of aggregation (T_{agg}) is measured using the back reflection optic of the nanoDSF instrument, Nanotemper Prometheus NT 48 (NanoTemper Technologies GmbH, Munich, Germany). For both meglumine salts, Meg-Glu and Meg-Asp, superior values are found in comparison to meglumine and sucrose alone or to their combined application.

On the basis of comparative experiments with sucrose and meglumine under otherwise identical conditions, the significant stabilizing effect of various meglumine salt forms as there are: meglumine-glutamate (Meg-Glu), meglumine-lactobionate (Meg-Lac) and meglumine aspartate (Meg-Asp) can be shown and benchmarked on the conformational (Tₘ) and colloidal (T_{agg}) stability of protein solutions of mAbA, mAbB and fusionA (Examples 2 A-F).

All model proteins were formulated at a rather high concentration of 50 mg/ml in 10 mM citrate buffer pH 5.

In all cases, the salt forms of meglumine show a superior stabilization potential in comparison to meglumine as such and in most cases also in comparison to the use of sucrose.

Accordingly, the present invention relates to stabilizing of proteins in solution, which includes the step of adding selected meglumine salts to protein solutions, especially to solutions of pharmaceutical active proteins. The stabilization according to the present invention may result in a long-term stabilization of the protein solution.

Herein, “long-term stabilization” is defined as follows: When the preparation is a protein solution, long-term stabilization means that the aggregate content is preferably less than 35% after two weeks of storage at 55° C.; alternatively, it is less than 10%, preferably less than 7%, after two weeks of storage at 40° C.; alternatively, it is less than 1% after two months of storage at 25° C.; alternatively, it is less than 2%, preferably 1% or less, after six months of storage at −20° C.

Target pharmaceutical compositions (proteins) to be stabilized according to the present invention may be proteins, including peptides, or other biopolymers, synthetic polymers, low molecular weight compounds, derivatives thereof, or complexes comprising a combination thereof. Preferred examples of the present invention are antibodies.

Target antibodies to be stabilized according to the present invention may be known antibodies, and may be any of whole antibodies, antibody fragments, modified antibodies, and minibodies or fusion proteins.

Known whole antibodies include IgGs (IgG1s, IgG2s, IgG3s, and IgG4s), IgIs, IgEs, IgMs, IgYs, and Such. The type of antibody is not particularly limited. Whole antibodies also include bispecific IgG antibodies (J. Immunol. Methods. 2001 Feb. 1; 248(1-2):7-15).

Antibodies prepared by methods known to those skilled in the art using novel antigens can also be targeted. In particular new antibodies can also be prepared by methods as disclosed in the known literature and by methods which are known to the person skilled in the art.

Target antibodies to be stabilized according to the present invention include antibody fragments and minibodies. The antibodies may be known antibodies or newly prepared antibodies. The antibody fragments and minibodies include antibody fragments which lack a portion of a whole antibody (for example, whole IgG). The antibody fragments and minibodies are not particularly limited, as long as they have the ability to bind to an antigen. Corresponding characterizations are known to the person skilled in the art and can be found in the literature known to him.

Essential to the present invention is that the stabilizing effect of the meglumine salts can be used for any pharmaceutically active protein solutions and that it is not limited to specific proteins. Advantageously, this stabilization can be carried out by known and tested means.

The antibodies to be used in the present invention may be modified antibodies. Modified antibodies may be conjugated antibodies obtained by linking with various molecules. Such as polyethylene glycol (PEG), radioactive substances, and toxins.

Furthermore, the modified antibodies include not only conjugated antibodies but also fusion proteins between an antibody molecule, antibody molecule fragment, or antibody-like molecule, and other proteins or peptides. Such fusion proteins include, but are not particularly limited to, fusion proteins between TNFC. and Fc (IntJ Clin Pract. 2005 January: 59(1): 114-8) and fusion proteins between IL-2 and scFv (J Immunol Methods. 2004 December; 295(1-2):49-56).

Furthermore, antibodies used in the present invention may also be antibody-like molecules. Antibody-like molecules include affibodies (Proc Natl AcadSci USA. 2003 Mar. 18; 100(6):3191-6) and ankyrins (Nat Biotechnol. 2004 May; 22(5):575-82), but are not particularly limited thereto.

The antibodies described above can be produced by methods known to those skilled in the art.

Herein, “adding” meglumine salts to proteins also means mixing meglumine with proteins. Herein, “mixing meglumine with proteins” may mean dissolving proteins in a meglumine salt containing solution. Herein, “stabilizing” means maintaining proteins in the natural state or preserving their activity.

Furthermore, when protein activity is enhanced upon addition of a stabilizer comprising a meglumine salt of the present invention as compared to the natural state or a control or when the degree of activity reduction due to aggregation during storage is decreased, the protein can also be assumed to be stabilized. Specifically, whether the activity of a protein, for example, an antibody molecule, is enhanced can be tested by assaying the activity of interest under the same conditions. Target antibody molecules to be stabilized include newly synthesized antibodies and antibodies isolated from organisms.

The activity of proteins of the present invention may be any activity, such as binding activity, neutralizing activity, cytotoxic activity, agonistic activity, antagonistic activity, and enzymatic activity. The activity is not particularly limited; however, the activity is preferably an activity that quantitatively and/or qualitatively alters or influences living bodies, tissues, cells, proteins, DNAs, RNAs, and such. Agonistic activities are especially preferred.

“Agonistic activity” refers to an activity that induces a change in some physiological activity by transducing a signal into cells and such, due to the binding of an antibody to an antigen such as a receptor. Physiological activities include, but are not limited to, for example, proliferation activity,

Survival activity, differentiation activity, transcriptional activity, membrane transportation activity, binding activity, proteolytic activity, phosphorylation/dephosphorylation activity, oxidation/reduction activity, transfer activity, nucleolytic activity, dehydration activity, cell death-inducing activity, and apoptosis-inducing activity.

The proteins, fusion proteins or antigens of the present invention are not particularly limited, and any antigen may be used.

Herein, “stabilizing proteins” means suppressing the increase of protein aggregate amount during storage by suppressing protein aggregation, and/or suppressing the increase in the amount of insoluble aggregates (precipitates) formed during storage, and/or maintaining protein function. Preferably, “stabilizing proteins” means suppressing the increase of the amount of protein aggregates formed during storage. The present invention relates to methods for suppressing protein aggregation, which comprise the step of adding selected meglumine salt to proteins. More specifically, the present invention relates to methods for suppressing aggregation of antibody molecules, which comprise the step of adding a selected meglumine salt to antibody molecules. Herein, aggregation refers to formation of multimers consisting of two or more antibody molecules via reversible or irreversible aggregation of proteins (antibody molecules).

Whether the aggregation is suppressed can be tested by measuring the content of antibody molecule aggregates by methods known to those skilled in the art, for example, sedimentation equilibrium method (ultracentrifugation method), osmometry, light scattering method, low-angle laser light scattering method, small angle X-ray scattering method, small-angle neutron scattering method, and gel filtration.

When the content of antibody aggregates during storage is reduced upon addition of a selected meglumine salt, the aggregation can be interpreted to be suppressed.

Herein, “stabilizing of peptide or protein or antibody molecules” includes stabilizing such molecules in solution preparations, freeze-dried preparations, but also spray-dried preparations, regardless of peptide, protein or antibody concentration and condition, and also includes stabilizing such molecules that are stored for a long term at a low temperatures or room temperature. Herein, low-temperature storage includes, for example, storage at −80° C. to 10° C. Thus, cryopreservation is also included in the storage means. Preferred low temperatures include, for example, −20° C. and 5° C., but are not limited thereto. Herein, room temperature storage includes, for example, storage at 15° C. to 30° C. Preferred room temperatures include, for example, 25° C., but are not limited thereto.

Solution preparations of proteins at high concentration can be formulated by methods known to those skilled in the art. For example, the membrane concentration method using a TFF membrane may be applied, as described by Shire, S. J. et al. in “Challenges in the development of high protein concentration formulations” (J. Pharm. Sc, 2004, 93(6), 1390-1402).

Freeze-drying can be carried out by methods known to those skilled in the art (Pharm. Biotechnol, 2002, 13, 109-33; Int. J. Pharm. 2000, 203(1-2), 1-60; Pharm. Res. 1997, 14(8), 969-75). For example, adequate amounts of solutions are aliquoted into vessels such as vials for freeze-drying. The vessels are placed in a freezing chamber or freeze-drying chamber, or immersed in a refrigerant, such as acetone/dry ice or liquid nitrogen, to achieve freeze-drying.

Furthermore, also spray-dried preparations can be formulated by methods known to those skilled in the art (J. Pharm. Sci. 1998 November; 87(11): 1406-11).

In particular, the present invention relates to compounds for stabilizing proteins and compounds for suppressing protein aggregation, which comprise selected meglumine salts. More specifically, the present invention relates to compounds for stabilizing antibody molecules and agents for suppressing aggregation of antibody molecules, which comprise at least one of special meglumine salts. The present invention also relates to compounds for stabilizing antibody molecules and agents for stabilizing antibody molecules in freeze-dried antibody preparations, which comprise at least one meglumine salt.

The agents of the present invention may comprise pharmaceutically acceptable carriers, such as preservatives and stabilizers. “Pharmaceutically acceptable carriers” means pharmaceutically acceptable materials that can be administered in combination with the above-described compounds. The carriers may be materials without a stabilization effect or materials that produce a synergistic or additive stabilization effect when used in combination with said meglumine salts. Such pharmaceutically acceptable materials may include, for example, sterile water, physiological saline, stabilizers, excipients, buffers, preservatives, detergents, chelating agents, and binders.

In the present invention, detergents include nonionic detergents. But preferably, the aim is to prepare formulations in which no detergents need to be added.

In the present invention, buffers include phosphate, citrate buffer, acetic acid, malic acid, tartaric acid, succinic acid, lactic acid, potassium phosphate, gluconic acid, caprylic acid, deoxycholic acid, salicylic acid, triethanolamine, fumaric acid, and other organic acids; and carbonic acid buffer, Tris buffer, histidine buffer, and imidazole buffer.

Solution preparations may be prepared by dissolving the agents in aqueous buffers known in the field of liquid preparations. The buffer concentration is in general 1 to 500 mM, preferably 5 to 100 mM, and more preferably 10 to 20 mM.

The agents of the present invention may also comprise other low molecular weight polypeptides; proteins such as serum albumin, gelatin, and immunoglobulin; amino acids; sugars and carbohydrates such as polysaccharides and monosaccharides; sugar alcohols and such.

Herein, amino acids include basic amino acids, for example, arginine, lysine, histidine, and ornithine, and inorganic salts of these amino acids (preferably in the form of hydrochlorides, and phosphates, namely phosphate amino acids). When free amino acids are used, the pH is adjusted to a preferred value by adding appropriate physiologically acceptable buffering substances, for example, inorganic acids, in particular hydrochloric acid, phosphoric acid, sulfuric acid, acetic acid, and formic acid, and salts thereof. In this case, the use of phosphate is particularly beneficial because it gives especially stable freeze-dried products. Phosphate is particularly advantageous when preparations do not substantially contain organic acids, such as malic acid, tartaric acid, citric acid. Succinic acid, and fumaric acid, or do not contain corresponding anions (malate ion, tartrate ion, citrate ion, succinate ion, fumarate ion, and such).

Preferred amino acids are arginine, lysine, histidine, and ornithine.

Furthermore, neutral amino acids, for example, isoleucine, leucine, glycine, serine, threonine, Valine, methionine, cysteine, and alanine; and aromatic amino acids, for example, phenylalanine, tyrosine, tryptophan, and its derivative, N-acetyl tryptophan may also be used.

Herein, sugars and carbohydrates such as polysaccharides and monosaccharides include, for example, dextran, glucose, fructose, lactose, xylose, mannose, maltose, sucrose, trehalose, and raffinose. Herein, sugar alcohols include, for example, mannitol, sorbitol, and inositol.

When the agents of the present invention are prepared as aqueous solutions for injection, the agents may be mixed with, for example, physiological saline, and/or isotonic solution containing glucose or other auxiliary agents (such as D-sorbitol, D-mannose, D-mannitol, and sodium chloride).

The aqueous solutions may be used in combination with appropriate solubilizing agents such as alcohols (ethanol and such), polyalcohols (propylene glycol, PEG, and such), or non-ionic detergents (polysorbate 80 and HCO-50). Preferably, however, aqueous solutions are used which contain no detergents.

The compositions of the invention may further comprise, if required, diluents, solubilizers, pH adjusters, soothing agents, sulfur-containing reducing agents, antioxidants, and such. Herein, sulfur-containing reducing agents include, for example, compounds comprising sulfhydryl groups, such as N-acetylcysteine, N-acetylhomocysteine, thioctic acid, thiodiglycol, thioethanolamine, thioglycerol, thiosorbitol, thioglycolic acid and salts thereof. Sodium thiosulfate, glutathione, and thioalkanoic acids having 1 to 7 carbon atoms. Preferably, however, compositions are used, wherein the number of different additives is kept as low as possible

Moreover, the antioxidants in the present invention include, for example, erythorbic acid, dibutylhydroxy toluene, butylhydroxyanisole, C-tocopherol, tocopherol acetate, L-ascorbic acid and salts thereof, L-ascorbic acid palmitate, L-ascorbic acid stearate, sodium hydrogen sulfite, sodium sulfite, triamyl gallate, propyl gallate, and chelating agents such as disodium ethylenediamine tetraacetic acid (EDTA), sodium pyrophosphate, and Sodium metaphosphate.

If required, the agents may be encapsulated in microcapsules (microcapsules of hydroxymethylcellulose, gelatin, polymethylmethacrylic acid or such) or prepared as colloidal drug delivery systems (liposome, albumin microspheres, microemulsion, nano-particles, nano-capsules, and such) (see “Remington's Pharmaceutical Science 16th edition”, Oslo Ed., 1980, and the like).

In particular, the present invention relates to pharmaceutical compositions comprising protein or peptide molecules, preferably antibody molecules, which are stabilized by at least one meglumine salt as specified above. The present invention also relates to pharmaceutical compositions comprising antibody molecules in which their aggregation is suppressed by meglumine salts. The present invention also relates to kits comprising the pharmaceutical compositions and pharmaceutically acceptable carriers. These kits can potentially be used for streamlined formulation screens e.g. by ready-to-use freeze-dried formulations sitting in a 96-well plate with subsequent DOE-analysis. With the help of a kit-device like this one can easily find out the optimum molar ratios between meglumine and its counterion for the respective active pharmaceutical ingredient e.g. a monoclonal antibody.

The pharmaceutical compositions and kits of the present invention may comprise pharmaceutically acceptable materials, in addition to the stabilized antibody molecules described above. Such pharmaceutically acceptable materials include the materials described above.

The formula (dosage form) of the pharmaceutical compositions of the present invention includes injections, freeze dried preparations, solutions, and spray-dried preparations, but is not limited thereto.

In general, the preparations of the present invention can be provided in containers with a fixed volume. Such as closed sterile plastic or glass vials, ampules, and injectors, or large volume containers, such as bottles. Prefilled syringes are preferred for the convenience of use.

Administration to patients is preferably a subcutaneous administration, such as an injection. Administration by injection includes, for example, intravenous injection, intramuscular injection, intraperitoneal injection, and subcutaneous injection, for systemic or local administration. The administration methods can be suitably selected according to the patient's age and symptoms.

The single-administration dose of a protein, peptide, or antibody can be selected, for example, in the range of 0.0001 mg to 500 mg/kg body weight. Alternatively, the dose can be selected, for example, from the range of 0.001 to 200,000 mg/patient. However, the dose and administration method of the present invention are not limited to those described above. The dose of a low molecular weight compound as an active ingredient may be in the range of 0.1 to 2000 mg/adult/day. But the dose and administration method of the present invention are not limited to those described above.

Freeze-dried or spray-dried preparations of the present invention can be made into solution preparations prior to use.

Thus, the present invention also provides kits comprising freeze-dried or spray-dried preparations of the present invention and pharmaceutically acceptable carriers.

There is no limitation on the type of pharmaceutically acceptable carrier, or on whether there is a combination of carriers or not, as long as the pharmaceutically acceptable carrier(s) allows formulation of freeze-dried or spray-dried preparations into solution preparations. The aggregation of antibody molecules in solution preparations can be suppressed by using a stabilizer of the present invention as a pharmaceutically acceptable carrier, or as part of pharmaceutically acceptable carrier.

Thus, the present invention relates to methods for producing pharmaceutical compositions comprising protein or peptide molecules, preferably antibody molecules, which comprise the step of adding a specific meglumine salt for stabilization. The present invention also relates to methods for producing pharmaceutical compositions comprising antibody molecules, which comprise the step of adding a meglumine salt to suppress the aggregation.

To be precise, the present invention relates to methods for producing pharmaceutical compositions comprising antibody molecules, which comprise the steps of:

(1) adding special meglumine salt to antibodies, each in a suitable formulation and
(2) formulating the mixture of (1) into solution preparations.

Furthermore, the present invention also relates to methods for producing pharmaceutical compositions comprising antibody molecules, which comprise the steps of:

(1) adding special meglumine salt to antibodies and
(2) freeze-drying the mixture of (1).

The formulation of solution preparations and of freeze dried preparations can be carried out by known methods and all prior art documents cited herein are incorporated by reference as part of the disclosure of the invention.

Aggregation of antibody molecules can be avoided by adding stabilizers comprising meglumine and selected counterions in a specially adjusted relationship to each other building the corresponding salts of the present invention. In the development of antibody formulations as pharmaceuticals, antibody molecules have to be stabilized so that the aggregation is suppressed to minimum during storage of preparations. The stabilizers of the present invention can stabilize antibody molecules and suppress aggregation even when the concentration of antibodies to be stabilized is very high. Thus, these stabilizers are very useful in producing antibody preparations. Furthermore, agents comprising a meglumine salt of the present invention also have the effect of stabilizing antibody molecules when the antibody molecules are formulated into liquid preparations or freeze-dried preparations. The stabilizers described here also have the effect of stabilizing antibody molecules against the stress imposed during the freeze-drying process in the formulation of freeze-dried preparations (Example 6). Advantageously the stabilizers of the present invention have the effect of stabilizing whole antibodies, antibody fragments, and minibodies, and thus may be widely used in production of antibody formulations for pharmaceutical application.

The pharmaceutical compositions of the present invention, which comprise antibody molecules stabilized by these meglumine salts of the present invention, are well-preserved, as compared to conventional antibody preparations, because the denaturation and aggregation of antibody molecules are suppressed. Therefore, the degree of activity loss by preservation as disclosed here is found to be very low.

The formulation of solution preparations and freeze drying can be carried out by the methods as described above and as disclosed in the following examples.

The present description enables one of ordinary skill in the art to practice the present invention comprehensively. Even without further comments, it is therefore assumed that a person of ordinary skill in the art will be able to utilise the above description in the broadest scope.

If anything is unclear, it is understood that the publications and patent literature cited and known to the artisan should be consulted. Accordingly, cited documents are regarded as part of the disclosure content of the present description and are incorporated herein by reference.

For better understanding and in order to illustrate the invention, examples are presented below which are within the scope of protection of the present invention. These examples also serve to illustrate possible variants.

Furthermore, it goes without saying to one of ordinary skill in the art that, both in the examples given and also in the remainder of the description, the component amounts present in the compositions always only add up to 100% by weight or mol %, based on the composition as a whole, and cannot exceed this percentage, even if higher values could arise from the percent ranges indicated. Unless indicated otherwise, % data are therefore % by weight or mol %, with the exception of ratios, which are shown in volume data.

### EXAMPLES

### Example 1: Stabilizing Effect of Meglumine-Glutamate and Meglumine-Aspartate Vs. Meglumine and Sucrose at Low Protein Concentrations (1 mg/ml) Against Isothermal Stress Analyzed Via Differential Scanning Fluorimetry

Examples 1A-C show a clear concentration dependent stabilizing effect of melgumine glutamate and meglumine aspartate towards the conformational stability (Tₘ) of mAbA, mAbB and the fusion protein fusionA.
At a concentration of 500 mM, the melting temperature (Tₘ) of mAbA which can be used as a predictive stability indicator for protein formulations is increased by 2.7° C. in the case of Meg-Glu and 2.2° C. in the case of Meg-Asp compared to meglumine.
Examples 1 D-E show a clear concentration dependent stabilizing effect of melgumine glutamate and meglumine aspartate towards the colloidal stability (T_{agg}), measured via the backreflection optic of the Nanotemper Prometheus of mAbA and mAbB
At a concentration of 500 mM, the onset temperature of aggregation (T_{agg}) of mAbA which can be used as a predictive stability indicator for protein formulations is increased by 2.3° C. in the case of Meg-Glu and 1.9° C. in the case of Meg-Asp compared to meglumine.

### Example 1 A) Stabilizing Effect of Meglumine-Glutamate and Meglumine Aspartate Vs. Meglumine and Sucrose Towards the Melting Temperature (Tₘ) of mAbA Formulated at 1 mg/ml in McIlvaine-Buffer pH 5 Shown in FIG. 1

### Buffer Preparation:

The pH 5 buffer preparation is done at room temperature and according to the McIlvaine buffer preparation (McIlvaine 1921) as described in literature. Solutions of 0.2 M di-sodium hydrogen phosphate (anhydrous) and 0.1 M citric acid (anhydrous) are prepared. 10.3 parts of the 0.2 M di-sodium hydrogen phosphate are added to 9.7 parts of 0.1 M citric acid solution. The pH value is checked and adjusted to 5.0 (+/−0.05) using ortho-phosphoric acid 85%, if necessary.

### Sample Preparation:

Excipient solutions of 100 mM, 250 mM and 500 mM of meglumine-glutamate, meglumine-aspartate, meglumine and sucrose are prepared in pH 5.0 McIlvaine buffer.
A concentrated protein solution of mAb A (app. 145 kDa), which is washed using the McIlvaine pH 5.0 buffer, is diluted to 1 mg/ml using the excipient solution.

### Nanodsf Method:

NanoDSF is a modified differential scanning fluorimetry method to determine protein stability employing intrinsic tryptophan or tyrosin fluorescence. Protein stability can be addressed by thermal unfolding experiments. The thermal stability of a protein is typically described by the ‘melting temperature’ or ‘Tₘ’, at which 50% of the protein population is unfolded, corresponding to the midpoint of the transition from folded to unfolded.
The sample volume is 10 μl and the heating rate 1° C./min, whereas the temperature ramp starts at 20° C. and lasts till 95° C.
Analysis is performed with the Nanototemper Prometheus NT 48 (NanoTemper Technologies GmbH, Munich, Germany)

### Example 1 B) Stabilizing Effect of Meglumine-Glutamate and Meglumine Aspartate Vs. Meglumine and Sucrose Towards the Melting Temperature (Tₘ) of mAbB Formulated at 1 mg/ml in McIlvaine-Buffer pH 5 Shown in FIG. 2

### Sample Preparation:

Excipient solutions of 100 mM, 250 mM and 500 mM of meglumine-glutamate, meglumine-aspartate, meglumine and sucrose are prepared in pH 5.0 McIlvaine buffer.
A concentrated protein solution of mAb B (app. 152 kDa), which is washed using the McIlvaine pH 5.0 buffer, is diluted to 1 mg/ml using the excipient solution.

The nanoDSF method is performed as described in Example 1 A).

### Example 1 C) Stabilizing Effect of Meglumine-Glutamate and Meglumine Aspartate Vs. Meglumine and Sucrose Towards the Melting Temperature (Tₘ) of the Fusion Protein fusionA Formulated at 1 mg/ml in McIlvaine-Buffer pH 5 Shown in FIG. 3

Sample Preparation:
Excipient solutions of 100 mM, 250 mM and 500 mM of meglumine-glutamate, meglumine-aspartate, meglumine and sucrose are prepared in pH 5.0 McIlvaine buffer.
A concentrated protein solution of fusionA (app. 71 kDa), which is washed using the McIlvaine pH 5.0 buffer, is diluted to 1 mg/ml using the excipient solution.

The nanoDSF method is performed as described in Example 1 A).

### Example 1 D): Stabilizing Effect of Meglumine-Glutamate and Meglumine Aspartate Vs. Meglumine and Sucrose Towards the Onset Temperature of Aggregation (T) for mAbA Formulated at 1 mg/ml in McIlvaine-Buffer pH 5 Shown in FIG. 4

### Sample Preparation:

Excipient solutions of 100 mM, 250 mM and 500 mM of meglumine-glutamate, meglumine-aspartate, meglumine and sucrose are prepared in pH 5.0 McIlvaine buffer.
A concentrated protein solution of mAbA (app. 145 kDa), which is washed using the McIlvaine pH 5.0 buffer, is diluted to 1 mg/ml using the excipient solution.

### T_{agg} Detection Method (Backreflection Optic):

The detection of temperature induced aggregation of proteins using the nanoDSF is achieved by measuring the back reflection of the emitted light beam which travels though the sample capillaries twice. If aggregation occurs, the light is scattered due to the formed aggregates and the intensity is reduced.
Analysis is performed with the Nanotemper Prometheus NT 48 (NanoTemper Technologies GmbH, Munich, Germany).

### Example 1 E): Stabilizing Effect of Meglumine-Glutamate and Meglumine Aspartate Vs. Meglumine and Sucrose Towards the Onset Temperature of Aggregation (T_{agg}) for mAbB Formulated at 1 mg/ml in McIlvaine-Buffer pH 5 Shown in FIG. 5

### Sample Preparation:

Excipient solutions of 100 mM, 250 mM and 500 mM of meglumine-glutamate, meglumine-aspartate, meglumine and sucrose are prepared in pH 5.0 McIlvaine buffer.
A concentrated protein solution of mAbB (app. 152 kDa), which is washed using the McIlvaine pH 5.0 buffer, is diluted to 1 mg/ml using the excipient solution.

T_{agg} detection method is applied as described in Example 1 D)

### Example 2: Stabilizing Effect of Meglumine-Glutamate, Meglumine-Aspartate and Meglumine-Lactobionate Vs. Meglumine and Sucrose at High Protein Concentrations (50 mg/ml) Against Isothermal Stress Analyzed Via Differential Scanning Fluorimetry

Examples 2A-C show a clear concentration dependent stabilizing effect of meglumine-glutamate (Meg-Glu), meglumine-lactobionate (Meg-Lac) and meglumine aspartate (Meg-Asp) towards the conformational stability of mAbA, mAbB and the fusion protein fusionA
At a concentration of 250 mM, the melting temperature (Tₘ) of mabA, which can be used as a predictive stability indicator for protein formulations, is increased by 2.3° C. in the case of Meg-Glu, 1.7° C. for Meg-Lac and Meg-Asp compared to meglumine.
Examples 2D-F show a clear concentration dependent stabilizing effect of melgumine glutamate, meglumine-lactobionate and meglumine aspartate towards the colloidal stability of mAbA, mAbB and fusionA
At a concentration of 250 mM, the onset temperature of aggregation (T_{agg}) of mAbA, which can be used as a predictive stability indicator for protein formulations, is increased by 2.5° C. in the case of Meg-Glu, 2.2° C. for Meg-Lac and 1.8° C. in the case of Meg-Asp compared to meglumine.

### Example 2 A) Stabilizing Effect of Meglumine-Glutamate (Meg-Glu), Meglumine-Lactobionate (Meg-Lac) and Meglumine Aspartate (Meg-Asp) Vs. Meglumine and Sucrose Towards the Melting Temperature (Tₘ) of mAbA Formulated at 50 mg/ml in 10 mM Citrate Buffer pH 5 Shown in FIG. 6

### Buffer Preparation:

A sufficient amount of tri-sodium citrate dihydrate is weighed into an appropriate flask for the preparation of a 10 mM Citrate buffer. The pH is adjusted with citric acid (anhydrous) until a pH value of 5.0 (+/−0.05) is reached.

### Sample Preparation:

Excipient stock solutions for meglumine-glutamate, meglumine-lactobionate, meglumine-aspartate, meglumine and sucrose with a concentration of 500 mM are prepared in 10 mM Citrate buffer pH 5.0.
A concentrated protein solution of mAb A (app. 145 kDa), which is washed using the 10 mM citrate buffer pH 5.0, is diluted to 50 mg/ml using the 500 mM excipient solution and the 10 mM citrate buffer pH 5.0 solution.

The nanoDSF method is performed as described in Example 1 A).

### Example 2 B) Stabilizing Effect of Meglumine-Glutamate (Meg-Glu), Meglumine-Lactobionate (Meg-Lac) and Meglumine Aspartate (Meg-Asp) Vs. Meglumine and Sucrose Towards the Melting Temperature (Tₘ) of mAbB Formulated at 50 mg/ml in 10 mM Citrate Buffer pH 5 Shown in FIG. 7

Sample preparation is performed as described in Example 2 A) using mAbB (152 kDa).
The nanoDSF method is performed as described in Example 1 A).

### Example 2 C) Stabilizing Effect of Meglumine-Glutamate (Meg-Glu), Meglumine-Lactobionate (Meg-Lac) and Meglumine Aspartate (Meg-Asp) Vs. Meglumine and Sucrose Towards the Melting Temperature (Tₘ) of fusionA Formulated at 50 mg/ml in 10 mM Citrate Buffer pH 5 Shown in FIG. 8

Sample preparation is performed as described in Example 2 A) using fusionA (71 kDa).
The nanoDSF method is performed as described in Example 1 A).

### Example 2 D) Stabilizing Effect of Meglumine-Glutamate (Meg-Glu), Meglumine-Lactobionate (Meg-Lac) and Meglumine Aspartate (Meg-Asp) Vs. Meglumine and Sucrose Towards the Onset Temperature of Aggregation (Taₘ) for mAbA Formulated at 50 mg/ml in 10 mM Citrate Buffer pH 5 Shown in FIG. 9

Sample preparation is performed as described in Example 2 A)
T_{agg} detection method is applied as described in Example 1 D).

### Example 2 E) Stabilizing Effect of Meglumine-Glutamate (Meg-Glu), Meglumine-Lactobionate (Meg-Lac) and Meglumine Aspartate (Meg-Asp) Vs. Meglumine and Sucrose Towards the Onset Temperature of Aggregation (Taₘ) for mAbB Formulated at 50 mg/ml in 10 mM Citrate Buffer pH 5 Shown in FIG. 10

Sample preparation is performed as described in Example 2 A) using mAbB (152 kDa).
T_{agg} detection method is applied as described in Example 1 D).

### Example 2 F) Stabilizing Effect of Meglumine-Glutamate (Meg-Glu), Meglumine-Lactobionate (Meg-Lac) and Meglumine Aspartate (Meg-Asp) Vs. Meglumine and Sucrose Towards the Onset Temperature of Aggregation (Taₘ) for fusionA Formulated at 50 mg/ml in 10 mM Citrate Buffer pH 5 Shown in FIG. 11

Sample preparation is performed as described in Example 2 A) using fusionA (71 kDa).
T_{agg} detection method is applied as described in Example 1 D).

### Example 3: Protein Stabilizing Effect of Meglumine-Glutamate Vs. Meglumine and Sucrose Against Isothermal Stress (SEC-Analysis)

Examples 3A-3C illustrate the decrease in monomer concentration of a monoclonal IgG1 antibody (mAbA) stored at a temperature of 60° C. for up to 180 minutes with varying concentrations of protein stabilizing additives.
At a concentration of 500 mM and a total stress time of 180 minutes at 60° C., the remaining mAbA concentration was 0.84 mg/ml in the case of meglumine-glutamate, 0.67 mg/ml for meglumine and 0.31 mg/ml for sucrose
This study clearly shows that the salt form of meglumine (here meglumine-glutamate) possesses a greater stabilization potential towards mAbA then the sole use of meglumine as well as sucrose.

### Example 3 A) Meglumine-Glutamate

Remaining protein-monomer concentration (shown in FIG. 12) [mg/ml] of mAbA at a concentration of 1 mg/ml formulated in a phosphate/citrate buffer (McIlvaine buffer) after isothermal stress at 60° C. for 180 minutes with varying concentrations of an equimolar mixture of meglumine and glutamate. The monomer content is detected with size exclusion chromatography (SEC).

### Conditions for SEC Analysis:

Eluent: 0.05 M Sodium phosphate/0.4 M Sodium perchlorate/pH 6.3
Pre-Column: Tosoh Bioscience TSKgel SuperSW Guard; 4 μm; 35×4.6 mm; Prod. No. 18762
Column: Tosoh Bioscience TSKgel SuperSW3000; 4 μm; 300×4.6 mm; Prod. No. 18675
Flow rate: 0.35 ml/min.
Detection wavelength: 214 nm

### Buffer Preparation:

The removal of salts or the exchange of buffers is accomplished using Amicon® Ultra-0.5 device by concentrating the sample, discarding the filtrate, then reconstituting the concentrate to the original sample volume with the desired solvent. The process of “washing out” is repeated 5 times.

The pH 5 buffer preparation is done according to the McIlvaine buffer preparation. Solutions of 0.2 M di-sodium hydrogen phosphate (anhydrous) and 0.1 M citric acid (anhydrous) are prepared. 10.3 parts of the 0.2 M di-sodium hydrogen phosphate are added to 9.7 parts of 0.1 M citric acid solution. The pH value is checked and adjusted to 5.0 (+/−0.05) using ortho-phosphoric acid 85%, if necessary.

Sample preparation is performed as follows:

Molecular weight of used components:
M(Meglumine)=195.21 g/mol
M(Glutamate)=187.13 g/mol
M(Sucrose)=342.30 g/mol
for 25 ml sample volume:

| | Meglumine/ | Glutamate |
|---|---|---|
| [mM] | [g] | [g] |
| 25 mM | 0.122 g | 0.117 g |
| 50 mM | 0.244 g | 0.234 g |
| 100 mM | 0.488 g | 0.468 g |
| 250 mM | 1.22 g | 1.17 g |
| 500 mM | 2.44 g | 2.34 g |

The appropriate amount of substance is weighed into a 25 ml glass flask. 20 ml of buffer is added into the flasks with the concentrations 25 mM, 50 mM, 100 mM and 250 mM, whereas 15 ml of buffer is added to the 500 mM concentration. The pH is adjusted to 5 using 85% H₃PO₄ or 1 mol/l NaOH (if necessary). Afterwards the solution is transferred to a 25 ml volumetric flask and filled up to the mark with buffer. The solutions are mixed thoroughly.

500 μl of antibody solution with a concentration of 1 mg/ml is prepared in the buffer solution for each concentration and transferred into 2 ml Eppendorf tubes.

The tubes with the antibody formulations are heated in an Eppendorf thermomixer. Every 60 min a sample of 50 μl is taken and analyzed using SEC. The final sample is taken after 180 min stress time.

### Example 3 B) Meglumine

Remaining protein-monomer concentration [mg/ml] of mAbA at a concentration of 1 mg/ml formulated in a phosphate/citrate buffer (McIlvaine buffer) after isothermal stress at 60° C. for 180 minutes with varying concentrations of meglumine shown in FIG. 13.

Sample preparation is performed as described in example 3 A) using the following masses:

Weight of meglumine (desired value) for 25 ml sample volume:

| (desired value) | Meglumin |
|---|---|
| [mM] | [g] |
| 25 | 0.122 |
| 50 | 0.244 |
| 100 | 0.488 |
| 250 | 1.22 |
| 500 | 2.44 |

### Example 3 C) Sucrose

Remaining protein-monomer concentration (shown in FIG. 14) [mg/ml] of mAbA at a concentration of 1 mg/ml formulated in a phosphate/citrate buffer (McIlvaine buffer) after isothermal stress at 60° C. for 180 minutes with varying concentrations of sucrose.

Sample preparation is performed as described in example 3 A) using the following masses:

Weight of sucrose for 25 ml sample volume:

| (desired value) | Sucrose |
|---|---|
| [mM] | [g] |
| 25 | 0.214 |
| 50 | 0.428 |
| 100 | 0.856 |
| 250 | 2.14 |
| 500 | 4.28 |

### Example 4: Protein Stabilizing Effect of Meglumine (Meg), Meglumine-Glutamate (Meg-Glu), Meglumine-Aspartate (Meg-Asp) and Meglumine-Lactobionate (Meg-Lacto) in a Controlled Long-Term Stability (Storage Conditions: 12 Weeks at 40° C./75% r.H.)

Example 4A (turbidity, shown in FIG. 15) and 4B (SEC, shown in FIG. 16) show an increase in stability for a fusion protein (fusionA).
The turbidity values for Meg-Glu, Meg-Lacto and Meg-Asp are significantly lower than the not stabilized samples containing only the buffer solution as well as sucrose.
The SEC content analysis reveals that the remaining monomer content of Meg-Glu, Meg-Lacto and Meg-Asp are significantly higher than the not stabilized samples containing only buffer or sucrose. Additionally, using only Meg as stabilizer exceeds significantly the content of sucrose after 12 weeks of storage.

### 10 mM Na-Citrate Solution pH 5:

2.94 g Na-Citrate*2 H₂O (M=294.10 g/mol) was weighed into an appropriate flask. 1 l of ultrapure water was added and the solution stirred until the substance was completely dissolved. The pH was adjusted to 5+/−0.05 using citric acid (solid). This solution was filtered using a 0.1 μm filter.

### Stock Solution 500 mM Meglumine:

9.76 g Meglumiune (M=195.21 g/mol) was weighed into an appropriate flask. App. 80 ml 10 mM Na-Citrate buffer pH 5 was added and the solution was stirred until the substance was completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Meglumine-Glutamate:

9.76 g Meglumiune (M=195.21 g/mol) and 9.36 g Na-glutamate (M=187.13 g/mol) were weighed into an appropriate flask. App. 80 ml 10 mM Na-Citrate buffer pH 5 was added and the solution was stirred until the substances were completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Meglumine-Aspartate:

9.76 g Meglumiune (M=195.21 g/mol) and 8.66 g Na-aspartate (M=173.10 g/mol) were weighed into an appropriate flask. App. 80 ml 10 mM Na-Citrate buffer pH 5 was added and the solution was stirred until the substances were completely dissolved. The pH was adjusted to 5+/−0.05 using citric acid (solid).

Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Meglumine-Lactobionate:

9.76 g Meglumiune (M=195.21 g/mol) and 17.92 g Lactobionic acid (M=358.30 g/mol) were weighed into an appropriate flask. App. 80 ml 10 mM Na-Citrate buffer pH 5 was added and the solution was stirred until the substances were completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Sucrose:

17.11 g Sucrose (M=342.29 g/mol) was weighed into an appropriate flask. App. 80 ml 10 mM Na-Citrate buffer pH 5 was added and the solution was stirred until the substance was completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Preparation of Sample Solutions for 3 Months Storage Stability

The storage conditions were set to 40° C. at 75% r.H. in a controlled climate cabinet. The sampling times were set to 0 weeks (initial value), 4 weeks, 8 weeks and 12 weeks.

The sample solutions containing fusionA as protein were prepared in 2R injection vials, which are closed using the appropriate plugs and aluminum clamps. Every sample vial was filled under laminar flow to reduce particle contamination.

A sample set of three samples containing 250 mM excipient, 50 mg/ml fusionA and Na-Citrate buffer pH 5 was prepared for each sampling time.

Additionally, a sample set of three samples containing only 10 mM Na-citrate buffer pH 5 with a fusionA concentration of 50 mg/ml was prepared as control sample.

The final volume for each sample was 500 μl consisting of Na-Citrate buffer pH 5, fusionA and excipient.

At each sampling time the samples were taken and stored in a freezer at −80° C. until the subsequent analysis was started.

### Example 5: Protein Stabilizing Effect of Meglumine (Meg), Meglumine-Glutamate (Meg-Glu), Meglumine-Aspartate (Meg-Asp) and Meglumine-Lactobionate (Meg-Lacto) in Isothermal Stress

Example 5A (turbidity, FIG. 17) and 5B (SEC, FIG. 18) show an increase in stability for a fusion protein (fusionA).
The SEC monomer content analysis reveals a significant concentration dependence regarding stabilizing effects for the excipients
At an excipient concentration of 100 mM meglumine and its salts show a significantly higher content than the not stabilized samples and those, which are stabilized with Sucrose.

### 10 mM Na-Citrate Solution pH 5:

2.94 g Na-Citrate*2 H₂O (M=294.10 g/mol) was weighed into an appropriate flask. 1 l of ultrapure water was added and the solution stirred until the substance was completely dissolved. The pH was adjusted to 5+/−0.05 using citric acid (solid). This solution was filtered using a 0.1 μm filter.

### Stock Solution 500 mM Meglumine:

9.76 g Meglumiune (M=195.21 g/mol) was weighed into an appropriate flask. App. 80 ml 10 mM Na-Citrate buffer pH 5 was added and the solution was stirred until the substance was completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Meglumine-Glutamate:

9.76 g Meglumiune (M=195.21 g/mol) and 9.36 g Na-glutamate (M=187.13 g/mol) were weighed into an appropriate flask. App. 80 ml 10 mM Na-Citrate buffer pH 5 was added and the solution was stirred until the substances were completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Meglumine-Aspartate:

9.76 g Meglumiune (M=195.21 g/mol) and 8.66 g Na-aspartate (M=173.10 g/mol) were weighed into an appropriate flask. App. 80 ml 10 mM Na-Citrate buffer pH 5 was added and the solution was stirred until the substances were completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Meglumine-Lactobionate:

9.76 g Meglumiune (M=195.21 g/mol) and 17.92 g Lactobionic acid (M=358.30 g/mol) were weighed into an appropriate flask. App. 80 ml 10 mM Na-Citrate buffer pH 5 was added and the solution was stirred until the substances were completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Stock Solution 500 mM Sucrose:

17.11 g Sucrose (M=342.29 g/mol) was weighed into an appropriate flask. App. 80 ml 10 mM Na-Citrate buffer pH 5 was added and the solution was stirred until the substance was completely dissolved. The pH was adjusted to 5+/−0.05 using citric acid (solid). Afterwards, the solution was transferred to a 100.0 ml volumetric graduated flask and filled to the mark with 10 mM Na-Citrate buffer pH 5 and mixed thoroughly. This solution was filtered using a 0.1 μm filter.

### Preparation of Sample Solutions for Isothermal Stress at 50° C. for 2 h

The isothermal stress was done using a drying oven adjusted to 50° C.

The sample solutions containing fusionA as protein were prepared in 2R injection vials, which are closed using the appropriate plugs and aluminum clamps. Every sample vial was filled under laminar flow to reduce particle contamination.

A sample set of three samples containing 100 mM and 250 mM excipient, 25 mg/ml fusionA and Na-Citrate buffer pH 5 was prepared for each sampling time.

Additionally, a sample set of three samples containing only 10 mM Na-Citrate buffer pH 5 with a fusionA concentration of 25 mg/ml was prepared as control sample.

The final volume for each sample was 300 μl consisting of Na-Citrate buffer pH 5, fusionA and excipient.

### Example 6: Protein Stabilizing Effect in Lyophilization of Meglumine (Meg) and its Salts in a Controlled Long-Term Stability (Storage Conditions: 3 Months at 40° C./75% r.H.)

Meglumine and its salts can be used in formulation relevant concentrations for lyophilization
Example 6A (Turbidity, FIG. 19) and example 6B (SEC-analysis, FIG. 20) shows an increase in stability for a mabA.
The turbidity values for Meglumine, Meg-HCl, Meg-Glu and Meg-Asp are significantly lower than the not stabilized samples containing only the buffer solution as well as sucrose and Meg-Lac.
The SEC content analysis reveals that the remaining monomer content of Meglumine, Sucrose, Meg-HCl, Meg-Lacto, Meg-Asp, Meg-Glu and Meg-Mes-based formulations are significantly higher than the not stabilized samples containing only buffer. Additionally, SEC results of Meg as stabilizer showed comparable monomer content with sucrose, Meg-HCl and Meg-Glu results after 12 weeks of storage.
The monomer content of mabA stabilized with 50 mM Meglumine-lactobionate, Meglumine-aspartate and Meglumine-mesylate was significantly higher compared to the other formulations (app. 90% of initial monomer content).

### Buffer and Excipient Stock Solution Preparation:

10 mM phosphate buffer pH 5:

1.42 g dibasic sodium phosphate anhydrous (M=141.96 g/mol) was weighed into an appropriate flask. 1 l of ultrapure water was added and the solution stirred until the substance was completely dissolved. The pH was adjusted to 5+/−0.05 using phosphoric acid 85 wt. % in H₂O or 1M NaOH. This solution was filtered using a 0.1 μm filter.

### Stock Solution 200 mM Meglumine:

3.9 g Meglumine (M=195.21 g/mol) was weighed into an appropriate flask. App. 80 ml 10 mM phosphate buffer pH 5 was added and the solution was stirred until the substance was completely dissolved. The pH was adjusted to 5+/−0.05 using phosphoric acid 85 wt. % in H₂O or 1M NaOH. Afterwards, the solution was transferred to a 100.0 ml volumetric graduated flask and filled to the mark with 10 mM phosphate buffer pH 5 and mixed thoroughly. This solution was filtered using a 0.1 μm filter.

### Stock Solution 200 mM Sucrose:

6.84 g sucrose (M=342.29 g/mol) is weighed into an appropriate flask. App. 80 ml 10 mM phosphate buffer pH 5 is added and the solution is stirred until the substance was completely dissolved. The pH is adjusted to 5+/−0.05 using phosphoric acid 85 wt. % in H₂O or 1M NaOH. Afterwards, the solution is transferred to a 100.0 ml volumetric graduated flask and filled up to the mark with 10 mM phosphate buffer pH 5 and mixed thoroughly. This solution is filtered using a 0.1 μm filter.

### Stock Solution 100 mM Meglumine-HCl:

1.95 g Meglumine (M=195.21 g/mol) were weighed into an appropriate flask. App. 80 ml 10 mM phosphate buffer pH 5 and 1 ml 1000 mM HCl were added and the solution was stirred until the substances were completely dissolved. The pH was adjusted to 5+/−0.05 using phosphoric acid 85 wt. % in H₂O or 1M NaOH. Afterwards, the solution was transferred to a 100.0 ml volumetric graduated flask and filled to the mark with 10 mM phosphate buffer pH 5 and mixed thoroughly. This solution was filtered using a 0.1 μm filter.

### Stock Solution 100 mM Meglumine-Lactobionate:

1.95 g Meglumine (M=195.21 g/mol) and 3.58 g Lactobionic acid (M=358.30 g/mol) were weighed into an appropriate flask. App. 80 ml 10 mM phosphate buffer pH 5 was added and the solution was stirred until the substances were completely dissolved. The pH was adjusted to 5+/−0.05 using phosphoric acid 85 wt. % in H₂O or 1M NaOH. Afterwards, the solution was transferred to a 100.0 ml volumetric graduated flask and filled to the mark with 10 mM phosphate buffer pH 5 and mixed thoroughly. This solution was filtered using a 0.1 μm filter.

### Stock Solution 100 mM Meglumine-Aspartate:

1.95 g Meglumine (M=195.21 g/mol) and 1.73 g Na-aspartate (M=173.10 g/mol) were weighed into an appropriate flask. App. 80 ml 10 mM phosphate buffer pH 5 was added and the solution was stirred until the substances were completely dissolved. The pH was adjusted to 5+/−0.05 using phosphoric acid 85 wt. % in H₂O or 1M NaOH. Afterwards, the solution was transferred to a 100.0 ml volumetric graduated flask and filled to the mark with 10 mM phosphate buffer pH 5 and mixed thoroughly. This solution was filtered using a 0.1 μm filter.

### Stock Solution 100 mM Meglumine-Glutamate:

1.95 g Meglumine (M=195.21 g/mol) and 1.87 g Na-glutamate (M=187.13 g/mol) were weighed into an appropriate flask. App. 80 ml 10 mM phosphate buffer pH 5 was added and the solution was stirred until the substances were completely dissolved. The pH was adjusted to 5+/−0.05 using phosphoric acid 85 wt. % in H₂O or 1M NaOH. Afterwards, the solution was transferred to a 100.0 ml volumetric graduated flask and filled to the mark with 10 mM phosphate buffer pH 5 and mixed thoroughly. This solution was filtered using a 0.1 μm filter.

Preparation of lyophilized samples for 3 months stability storage:

A concentrated protein solution of mAbA (app. 145 kDa), which was washed using the 10 mM phosphate buffer pH 5.0, was diluted using the excipient stock solution or buffer to the desired concentration (50 mg/ml mabA) and formulation (25 mM and 50 mM for mixture of Meglumin and counter ion; 50 mM and 100 mM for Meglumine and Sucrose).

The sample solutions containing mabA were prepared in 2R injection vials, which are closed using the appropriate plugs. Every sample vial was filled under laminar flow to reduce particle contamination.

A sample set of two samples containing excipient, 50 mg/ml mabA and 10 mM phosphate buffer pH 5 was prepared for each sampling time.

Additionally, a sample set of two samples containing only 10 mM phosphate buffer pH 5 with a mabA concentration of 50 mg/ml was prepared as control sample for each sampling time.

The final volume for each sample was 1 ml consisting of 10 mM phosphate buffer pH 5, mabA and excipient.

The samples were then lyophilized using Martin Christ freeze dryer Epsilon 2-12D.

Freeze-drying is performed using the following protocol:

| | | Time | Temp. | Vacuum | Pressure |
|---|---|---|---|---|---|
| Steps | Phase | (h:m) | (° C.) | (mBar) | (mbar) |
| 1 | Starting value | —:— | 20 | OFF | OFF |
| 2 | Freezing | 01:00 | 5 | OFF | OFF |
| 3 | Freezing | 00:55 | −50 | OFF | OFF |
| 4 | Freezing | 04:30 | −50 | OFF | OFF |
| 5 | Preparation | 00:30 | −50 | OFF | OFF |
| 6 | Main drying | 00:01 | −50 | 0.05 | OFF |
| 7 | Main drying | 01:00 | −50 | 0.05 | 0.25 |
| 8 | Main drying | 01:00 | −45 | 0.05 | 0.25 |
| 9 | Main drying | 08:00 | −45 | 0.05 | 0.25 |
| 10 | Main drying | 01:00 | −40 | 0.05 | 0.25 |
| 11 | Main drying | 40:00 | −40 | 0.05 | 0.25 |
| 12 | Main drying | 01:00 | −35 | 0.05 | 0.25 |
| 13 | Main drying | 15:00 | −35 | 0.05 | 0.25 |
| 14 | Main drying | 01:00 | −30 | 0.05 | 0.25 |
| 15 | Main drying | 08:00 | −30 | 0.05 | 0.25 |
| 16 | Main drying | 01:00 | −20 | 0.05 | 0.25 |
| 17 | Main drying | 04:00 | −20 | 0.05 | 0.25 |
| 18 | Main drying | 07:30 | 25 | 0.009 | 0.25 |
| 19 | Post drying | 00:01 | 25 | 0.003 | 1.65 |
| 20 | Post drying | 10:00 | 25 | 0.003 | 1.65 |

After the lyophilization steps, the samples were closed using the appropriate aluminum clamps and stored in a controlled climate cabinet with storage condition of 40° C. at 75% r.H. The sampling times were set to 0 weeks (initial value), 4 weeks, 9 weeks and 12 weeks after lyophilization. At each sampling time the lyophilized samples were taken and reconstituted with 1 ml milli-Q-water for analysis.

### Example 7: Protein Stabilizing Effect of Meglumine-Glutamate (Meg-Glu)

vs. Meglumine and Sucrose at pH 7
The tested meglumine salt (Meg-Glu) can stabilize mabB better than sucrose or meglumine alone at pH 7 which can be visualized in the Tm—but especially in the Tagg values
Formulating proteins close to the physiological pH˜7.4 would be desirable since this would reduce injection pain, however most proteins have a pl close to that range and therefore need to be formulated close to pH 5-6
Surprisingly, adding Meg-Glu to a solution significantly improves the colloidal stability (represented via Tagg) compared to Meglumine alone and Sucrose
The Tm values were increasing from pH 5 to pH 7 for all tested conditions whereas the highest value for Tm was reached using Meg-Glu as excipient
   Buffer and excipient Stock Solution Preparation:
   6.67 mM phosphate solution

0.758 g of Na2HPO4 was weighed in to an appropriate flask. 800 ml of ultrapure water was added and the solution was stirred until the substance was completely dissolved. The final solution was filtered through a 0.1 μm filter.

3.33 mM citrate solution

0.320 g of citric acid was weighed in to an appropriate flask. 500 ml of ultrapure water was added and the solution was stirred until the substance was completely dissolved. The final solution was filtered through a 0.1 μm filter.

Preparation of phosphate-citrate buffer pH 5

257.5 ml of the 6.67 mM phosphate solution and 242.5 ml of the 3.33 mM citrate solution were filled into an appropriate flask and mixed thoroughly. The pH of the solution was adjusted to 5+/−0.05 using 1 M phosphoric acid or 1 M NaOH.

Preparation of phosphate-citrate buffer pH 7

411.7 ml of the 6.67 mM phosphate solution and 88.3 ml of the 3.33 mM citrate solution were filled into an appropriate flask and mixed thoroughly. The pH of the solution was adjusted to 7+/−0.05 using 1 M phosphoric acid or 1 M NaOH.

### Stock Solution 500 mM Meglumine pH 5:

1.95 g Meglumiune (M=195.21 g/mol) was weighed into an appropriate flask. App. 15 ml phosphate-citrate buffer pH 5 was added and the solution was stirred until the substance was completely dissolved. The pH was adjusted to 5+/−0.05 using 1 M phosphoric acid or 1 M NaOH. Afterwards, the solution was transferred to a 20.0 ml volumetric graduated flask and filled to the mark with phosphate-citrate buffer pH 5 and mixed thoroughly.

### Stock Solution 500 mM Meglumine-Glutamate pH 5:

1.95 g Meglumiune (M=195.21 g/mol) and 1.87 g Na-glutamate (M=187.13 g/mol) were weighed into an appropriate flask. App. 15 ml phosphate-citrate buffer pH 5 was added and the solution was stirred until the substances were completely dissolved. The pH was adjusted to 5+/−0.05 using 1 M phosphoric acid or 1 M NaOH. Afterwards, the solution was transferred to a 20.0 ml volumetric graduated flask and filled to the mark with phosphate-citrate buffer pH 5 and mixed thoroughly.

### Stock Solution 500 mM Sucrose pH 5:

3.42 g Sucrose (M=342.29 g/mol) was weighed into an appropriate flask. App. 15 ml phosphate-citrate buffer pH 5 was added and the solution was stirred until the substance was completely dissolved. The pH was adjusted to 5+/−0.05 using 1 M phosphoric acid or 1 M NaOH. Afterwards, the solution was transferred to a 20.0 ml volumetric graduated flask and filled to the mark with phosphate-citrate buffer pH 5 and mixed thoroughly.

### Stock Solution 500 mM Meglumine pH 7:

1.95 g Meglumiune (M=195.21 g/mol) was weighed into an appropriate flask. App. 15 ml phosphate-citrate buffer pH 7 was added and the solution was stirred until the substance was completely dissolved. The pH was adjusted to 7+/−0.05 using 1 M phosphoric acid or 1 M NaOH. Afterwards, the solution was transferred to a 20.0 ml volumetric graduated flask and filled to the mark with phosphate-citrate buffer pH 7 and mixed thoroughly.

### Stock Solution 500 mM Meglumine-Glutamate pH 7:

1.95 g Meglumiune (M=195.21 g/mol) and 1.87 g Na-glutamate (M=187.13 g/mol) were weighed into an appropriate flask. App. 15 ml phosphate-citrate buffer pH 7 was added and the solution was stirred until the substances were completely dissolved. The pH was adjusted to 7+/−0.05 using 1 M phosphoric acid or 1 M NaOH. Afterwards, the solution was transferred to a 20.0 ml volumetric graduated flask and filled to the mark with phosphate-citrate buffer pH 7 and mixed thoroughly.

### Stock Solution 500 mM Sucrose pH 7:

3.42 g Sucrose (M=342.29 g/mol) was weighed into an appropriate flask. App. 15 ml phosphate-citrate buffer pH 7 was added and the solution was stirred until the substance was completely dissolved. The pH was adjusted to 7+/−0.05 using 1 M phosphoric acid or 1 M NaOH. Afterwards, the solution was transferred to a 20.0 ml volumetric graduated flask and filled to the mark with phosphate-citrate buffer pH 7 and mixed thoroughly.

### Sample Preparation

The mabB stock solution was diluted with a sufficient volume of the excipient stock solution and phosphate citrate buffer of the corresponding pH value to reach a final excipient concentration of 50 mM/250 mM and 50 mg/ml mabB.

The Tm/Tagg values for mabB at 50 mg/ml for the pH 5 and the pH 7 solutions were analyzed using the Nanotemper Prometheus NT 48 (NanoTemper Technologies GmbH, Munich, Germany). Triplicate measurements of the same solution were carried out.

### Example 7

Tm/Tagg values for mabB 50 mg/ml stabilized with 50 mM/250 mM meglumine at pH 5 and pH 7 shown in FIGS. 21 and 22.

Tm/Tagg values for mabB 50 mg/ml stabilized with 50 mM/250 mM sucrose at pH 5 and pH 7 shown in FIGS. 23 and 24.

Tm/Tagg values for mabB 50 mg/ml stabilized with 50 mM/250 mM meglumine-glutamate at pH 5 and pH 7 shown in FIGS. 25 and 26.

### Example 8: Sub Visual Particle Measurement According to Pharm. Eur./USP Using the Fluid Imaging FlowCam 8100 of 12 Weeks Stability Samples with 25 mg/ml fusionA Stored at 25° C./60% r.H. And 2-8° C.

The particle measurement is done during a stability study set up with the protein fusionA with buffers of 10 mM Na-citrate pH 5.0 and 10 mM histidine pH 7.0. The target concentration for fusionA was 25 mg/ml and the following formulations are prepared with the buffer solutions pH 5.0 and pH 7.0:

10 mM buffer
50 mM/200 mM trehalose
50 mM/200 mM meglumine
25 mM/100 mM meglumine+25 mM/100 mM Na-glutamate
25 mM/100 mM meglumine+25 mM/100 mM Na-aspartate
25 mM/100 mM meglumine+25 mM/100 mM lactobionic acid
50 mM/200 mM sucrose
25 mM/100 mM arginine+25 mM/100 mM Na-glutamate
Preparation of 10 mM citrate buffer pH 5.0

1.92 g citric acid/liter is weighed into a flask and filled with the appropriate volume of ultra-pure water. The pH is adjusted to 5.0 (+/−0.05) using sodium hydroxide solution. The final solution is filtered through a 0.22 μm filter and stored at 2-8° C.

Preparation of 10 mM histidine buffer pH 7.0

1.55 g histidine/liter is weighed into a flask and filled with the appropriate volume of ultra-pure water. The pH is adjusted to 7.0 (+/−0.05) using hydrochloric acid solution. The final solution is filtered through a 0.22 μm filter and stored at 2-8° C.

### Preparation of Excipient Stock Solutions

400 mM Trehalose [20.54 g trehalose (342.30 g/mol)] is weighed into two 200 ml flasks. 150 ml of buffer pH 5.0 was added to one flask, 150 ml of buffer pH 7.0 is added to the other one. The solution is stirred until the substance is completely dissolved and the pH was adjusted to 5.0 and 7.0 respectively, if necessary. The flasks are filled to the mark using the appropriate buffer solution. The solutions are filtered through a 0.22 μm filter and stored in a fridge at 2-8° C.

400 mM Sucrose [20.54 g sucrose (342.30 g/mol)] is weighed into two 200 ml flasks. 150 ml of buffer pH 5.0 is added to one flask, 150 ml of buffer pH 7.0 is added to the other one. The solution is stirred until the substance is completely dissolved and the pH is adjusted to 5.0 and 7.0 respectively, if necessary. The flasks are filled to the mark (150 ml) using the appropriate buffer solution. The solutions are filtered through a 0.22 μm filter and stored in a fridge at 2-8° C.

400 mM Meglumine [11.71 g meglumine (195.22 g/mol)] is weighed into two 200 ml flasks. 100 ml of buffer pH 5.0 is added to one flask, 100 ml of buffer pH 7.0 is added to the other one. The solution is stirred until the substance is completely dissolved and the pH is adjusted to 5.0 and 7.0 respectively. The solutions are transferred to separate graduated flasks, which are then filled to the mark (150 ml) with the appropriate buffer solution and mixed thoroughly. The solutions are filtered through a 0.22 μm filter and stored in a fridge at 2-8° C.

### 200 mM Meglumine and 200 mM Na-Glutamate

5.85 g meglumine (195.22 g/mol) and 5.61 g Na-glutamate (187.13 g/mol) are weighed into two 200 ml flasks. 100 ml of buffer pH 5.0 is added to one flask, 100 ml of buffer pH 7.0 is added to the other one. The solution is stirred until the substances are completely dissolved and the pH is adjusted to 5.0 and 7.0 respectively. The solutions are transferred to separate graduated flasks, which are then filled to the mark (150 ml) with the appropriate buffer solution and mixed thoroughly. The solutions are filtered through a 0.22 μm filter and stored in a fridge at 2-8° C.

### 200 mM Meglumine and 200 mM Na-aspartate

5.85 g meglumine (195.22 g/mol) and 5.19 g Na-aspartate (173.10 g/mol) are weighed into two 200 ml flasks. 100 ml of buffer pH 5.0 is added to one flask, 100 ml of buffer pH 7.0 is added to the other one. The solution is stirred until the substances are completely dissolved and the pH is adjusted to 5.0 and 7.0 respectively. The solutions are transferred to separate graduated flasks, which are then filled to the mark (150 ml) with the appropriate buffer solution and mixed thoroughly. The solutions are filtered through a 0.22 μm filter and stored in a fridge at 2-8° C.

200 mM Meglumine+200 mM lactobionic acid

5.85 g meglumine (195.22 g/mol) and 10.75 g lactobionic acid (358.30 g/mol) are weighed into two 200 ml flasks. 100 ml of buffer pH 5.0 is added to one flask, 100 ml of buffer pH 7.0 is added to the other one. The solution is stirred until the substances are completely dissolved and the pH is adjusted to 5.0 and 7.0 respectively. The solutions are transferred to separate graduated flasks, which are then filled to the mark (150 ml) with the appropriate buffer solution and mixed thoroughly. The solutions are filtered through a 0.22 μm filter and stored in a fridge at 2-8° C.

### 200 mM Arginine+200 mM Na-Glutamate

5.23 g arginine (174.20 g/mol) and 5.61 g Na-glutamate (187.13 g/mol) are weighed into two 200 ml flasks. 100 ml of buffer pH 5.0 is added to one flask, 100 ml of buffer pH 7.0 is added to the other one. The solution is stirred until the substances are completely dissolved and the pH was adjusted to 5.0 and 7.0 respectively. The solutions are transferred to separate graduated flasks, which are then filled to the mark (150 ml) with the appropriate buffer solution and mixed thoroughly. The solutions are filtered through a 0.22 μm filter and stored in a fridge at 2-8° C.

### Protein Stock Solution

The stability study used fusionA as model protein at two pH values (5.0/7.0). Therefore, a protein stock solution with these pH values are used.
10 mM citric acid buffer pH 5.0: fusionA c=134.4 mg/ml
10 mM histidine buffer pH 7.0: fusionA c=172.4 mg/ml

### Sample Preparation

The stability study at 25° C./60% r. H. is carried out not only with liquid sample but also freeze-dried samples are prepared in a Martin Christ freeze dryer Epsilon 2-12D.

Sample volumes for preparation of fusionA samples for stability study at pH 5.0 shown in FIG. 27.

According to the table below all excipient solutions for the pH 5.0 condition are pipetted and mixed with care but thoroughly.

| | Target | | | | |
|---|---|---|---|---|---|
| Target | Concen- | | | | |
| Concen- | tration | Volume | Volume | Volume | |
| tration | Excipient | Protein | Excipient | Buffer | Sum |
| of Protein | in Sol. | Stock Sol. | Stock Sol. | Sol. | Volumina |
| [mg/ml] | [mM] | [μl] | [μl] | [μl] | [μl] |
| 25.0 | 50.0 | 10200.0 | 6900.0 | 37900.0 | 55000.0 |
| 25.0 | 200.0 | 10200.0 | 27500.0 | 17300.0 | 55000.0 |

Sample volumes for preparation of fusionA samples for stability study at pH 7.0 shown in FIG. 28.

According to the table below all excipient solutions for the pH 7.0 condition are pipetted and mixed with care but thoroughly.

| | Target | | | | |
|---|---|---|---|---|---|
| Target | Concen- | | | | |
| Concen- | tration | Volume | Volume | Volume | |
| tration | Excipient | Protein | Excipient | Buffer | Sum |
| of Protein | in Sol. | Stock Sol. | Stock Sol. | Sol. | Volumina |
| [mg/ml] | [mM] | [μl] | [μl] | [μl] | [μl] |
| 25.0 | 50.0 | 8000.0 | 6900.0 | 40100.0 | 55000.0 |
| 25.0 | 200.0 | 8000.0 | 27500.0 | 19500.0 | 55000.0 |

Finally, 15 excipient solutions for each pH condition are obtained.

The preparation of the stability samples is done by pipetting the appropriate solution into 2R vials needed for the stability study. The 2R vials are closed with either a lyophilization stopper or a standard stopper. The 2R vials with the lyophilization stoppers are freeze dried. All vials are closed with an aluminum crimp cap.

After the sample preparation process the samples are stored either in a climate cabinet at 25° C./60 r. H. or a fridge at 2−8° C.

Conditions for freeze-drying shown in FIG. 29.

A1) Liquid samples of 25 mg/ml fusionA pH 5.0 stored at 25° C./60% r. H.

Sub-visual particles after 0, 4, 8 or 12 weeks of storage of 25 mg/ml fusionA liquid formulation pH 5.0 at 25° C./60% r. H. shown in FIG. 30 (>10 μm) and FIG. 31 (>25 μm)

Comparison of 50 mM and 200 mM sucrose and meglumine-glutamate in terms of sub-visual particles for 25 mg/ml fusionA liquid formulation pH 5.0 stored for 0, 4, 8 or 12 weeks at 25° C./60% r.H. shown in FIG. 32.

The FIG. 30 and FIG. 31 as well as the FIG. 32 show that the amount of sub-visual particles with the samples stabilized with meglumine-glutamate are in most cases below the values or at least comparable with the standard stabilizer for proteins sucrose. Therefore, it can be concluded that meglumine-glutamate stabilizes at least as good as sucrose with a better tendency for lower particle values.

There is no significant trend or change in pH-value, osmolality and turbidity visible during the 12 weeks stability study. Therefore, there is no obvious evidence that the formulations undergo decomposition during the storage.

A2) Liquid samples of 25 mg/ml fusionA pH 7.0 stored at 25° C./60% r. H.

Sub-visual particles after 0, 4, 8 and 12 weeks of storage of 25 mg/ml fusionA liquid formulation pH 7.0 at 25° C./60% r.H. shown in FIG. 33 (>10 μm) and FIG. 34 (>25 μm).

The Comparison of 50 mM and 200 mM sucrose and meglumine-glutamate in terms of sub-visual particles for 25 mg/ml fusionA liquid formulation pH 7.0 stored for 0, 4, 8 or 12 weeks at 25° C./60% r. H. is shown in FIG. 35.

For the liquid formulation of the protein fusionA in 10 mM histidine buffer pH 7.0 the same trend is visible as it is shown for the formulation in buffer pH 5.0. FIG. 33 and FIG. 34 as well as FIG. 35 show that the amount of sub-visual particles is for meglumine-glutamate in most cases below the value of the standard protein stabilizer substance sucrose and therefore the same conclusion can be drawn: meglumine-glutamate stabilizes at least as good as sucrose with a better tendency for lower particle values.

There is no significant trend or change in pH-value, osmolality and turbidity visible during the 12 weeks stability study. Therefore, there is no obvious evidence that the formulations undergo decomposition during the storage.

B1) Freeze-dried samples of 25 mg/ml fusionA pH 5.0 stored at 25° C./60% r. H.

Sub-visual particles after 0, 4 and 12 weeks of storage of 25 mg/ml fusionA freeze-dried formulation pH 5.0 at 25° C./60% r.H. shown in FIG. 36 (>10 μm) and FIG. 37 (>25 μm)

FIG. 40: Comparison of 50 mM and 200 mM sucrose and meglumine-glutamate in terms of sub-visual particles for 25 mg/ml fusionA freeze-dried formulation pH 5.0 stored for 0, 4 and 12 weeks at 25° C./60% r.H. shown in FIG. 38.

As shown in FIG. 36, FIG. 37 and FIG. 38 the freeze-dried formulation of the protein fusionA in 10 mM citrate buffer pH 5.0 is the amount of sub-visual particles for meglumine-glutamate in most cases below the value of the standard protein stabilizer substance sucrose. Therefore, it can be concluded that meglumine-glutamate stabilizes at least as good as sucrose.

There is no significant trend or change in pH-value, osmolality and turbidity visible during the 12 weeks stability study. Therefore, there is no obvious evidence that the formulations undergo decomposition during the storage.

B2) Freeze-dried samples of 25 mg/ml fusionA pH 7.0 stored at 25° C./60% r. H.

Sub-visual particles after 0, 4 and 12 weeks of storage of 25 mg/ml fusionA freeze-dried formulation pH 7.0 at 25° C./60% r. H. shown in FIG. 39 (>10 μm) and FIG. 40 (>25 μm)

Comparison of 50 mM and 200 mM sucrose and meglumine-glutamate in terms of sub-visual particles for 25 mg/ml fusionA freeze-dried formulation pH 7.0 stored for 0, 4 and 12 weeks at 25° C./60% r. H. shown in FIG. 41.

For the freeze-dried formulation of the protein fusionA in 10 mM histidine buffer pH 7.0 the same trend is visible as it was shown for the formulation in buffer pH 5.0. The amount of sub-visual particles for meglumine-glutamate is in most cases in the same range of the standard protein stabilizer substance sucrose and therefore the same conclusion can be drawn: meglumine-glutamate stabilizes at least as good as sucrose.

There is no significant trend or change in pH-value, osmolality and turbidity visible during the 12 weeks stability study. Therefore, there is no obvious evidence that the formulations undergo decomposition during the storage.

C1) Liquid samples of 25 mg/ml fusionA pH 5.0 stored at 2-8° C.

Sub-visual particles after 12 weeks of storage of 25 mg/ml fusionA freeze-dried formulation pH 5.0 at 2-8° C. shown in FIG. 42 (>10 μm) and FIG. 43 (>25 μm).

Comparison of 50 mM and 200 mM sucrose and meglumine-glutamate in terms of sub-visual particles for 25 mg/ml fusionA liquid formulation pH 5.0 stored at 2-8° C. shown in FIG. 44.

For the liquid formulation of the protein fusionA in 10 mM citrate buffer pH 5.0 stored at 2-8° C. it was shown that the amount of sub-visual particles for meglumine-glutamate in most cases is below the value of the standard protein stabilizer substance sucrose and therefore it can be concluded: meglumine-glutamate stabilizes at least as good as sucrose with a better tendency for lower particle values.

There is no significant trend or change in pH-value, osmolality and turbidity visible during the 12 weeks stability. Therefore, there is no obvious evidence that the formulations undergo decomposition during the storage.

C2) Liquid samples of 25 mg/ml fusionA pH 7.0 stored at 2-8° C.

Sub-visual particles after 12 weeks of storage of 25 mg/ml fusionA freeze-dried formulation pH 7.0 at 2-8° C. shown in FIG. 45 (>10 μm) and FIG. 46 (>25 μm).

Comparison of 50 mM and 200 mM sucrose and meglumine-glutamate in terms of sub-visual particles for 25 mg/ml fusionA liquid formulation pH 7.0 stored at 2-8° C. shown in FIG. 47.

As FIG. 45, FIG. 46 and FIG. 47 for the liquid formulation of the protein fusionA in 10 mM histidine buffer pH 7.0 stored at 2-8° C. the same trend is visible as it is shown for the formulation in buffer pH 5.0. The amount of sub-visual particles for meglumine-glutamate is in most cases below the value of the standard protein stabilizer substance sucrose and therefore the same conclusion can be drawn: meglumine-glutamate stabilizes at least as good as sucrose with a better tendency for lower particle values at 200 mM.

There is no significant trend or change in pH-value, osmolality and turbidity visible during the 12 weeks stability. Therefore, there is no obvious evidence that the formulations undergo decomposition during the storage.

### Example 9: SEC Measurement of 12 Weeks Stability Samples with 25 mg/ml fusionA Stored at 25° C./60% r.H. And 2-8° C.

A1) Liquid samples of 25 mg/ml fusionA pH 5.0 stored at 25° C./60% r.H.

SEC results for content fusionA monomer after 0, 4, 8 and 12 weeks of storage of 25 mg/ml fusionA liquid formulation pH 5.0 at 25° C./60% r. H. shown in FIG. 48.

SEC results for purity fusionA monomer after 0, 4, 8 and 12 weeks of storage of 25 mg/ml fusionA liquid formulation pH 5.0 at 25° C./60% r. H. shown in FIG. 49.

At 25° C./60% r. H. the liquid formulations of fusionA with 10 mM citrate buffer pH 5.0 show a slight reduction in content and purity for every formulation. As the well-established substances sucrose, trehalose and arginine-glutamate are not superior over the meglumine formulations it can be concluded that the meglumine stabilized protein formulations are at least as stable as the well-known substances.

A2) Liquid samples of 25 mg/ml fusionA pH 7.0 stored at 25° C./60% r. H.

SEC results for content fusionA monomer after 0, 4, 8 and 12 weeks of storage of 25 mg/ml fusionA liquid formulation pH 7.0 at 25° C./60% r. H. shown in FIG. 50.

SEC results for purity fusionA monomer after 0, 4, 8 and 12 weeks of storage of 25 mg/ml fusionA liquid formulation pH 7.0 at 25° C./60% r. H. shown in FIG. 51.

The liquid formulations of fusionA with 10 mM histidine buffer pH 7 show significant reductions in content and monomer purity. Additionally, it can be shown that meglumine-glutamate and in a slight lesser manner meglumine-lactobionic acid stabilize the protein formulation at least in a comparable way as the well-established substances sucrose and trehalose.

B1) Freeze-dried samples of 25 mg/ml fusionA pH 5.0 stored at 25° C./60% r. H.

SEC results for content fusionA monomer after 0, 4 and 12 weeks of storage of 25 mg/ml fusionA freeze-dried formulation pH 5.0 at 25° C./60% r. H. shown in FIG. 52.

SEC results for purity fusionA monomer after 0, 4 and 12 weeks of storage of 25 mg/ml fusionA freeze-dried formulation pH 5.0 at 25° C./60% r.H. shown in FIG. 53.

The results of the freeze-dried formulations of fusionA in 10 mM citrate buffer pH 5.0 stored at 25° C./60% r. H. show only for the formulation without any excipient clear reductions in terms of content and purity. The excipient stabilized samples show slight reductions in content and purity but a distinction between the tested formulations is not possible as they are mostly on the same level.

B2) Freeze-dried samples of 25 mg/ml fusionA pH 7.0 stored at 25° C./60% r. H.

SEC results for content fusionA monomer after 0, 4 and 12 weeks of storage of 25 mg/ml fusionA freeze-dried formulation pH 7.0 at 25° C./60% r. H. shown in FIG. 54.

SEC results for purity fusionA monomer after 0, 4 and 12 weeks of storage of 25 mg/ml fusionA freeze-dried formulation pH 7.0 at 25° C./60% r. H. shown in FIG. 55.

The freeze-dried formulations of fusionA at pH 7.0 show a slight reduction in content after 12 weeks of storage and only the not stabilized formulation show a significant reduction in monomer purity.

C1) Liquid samples of 25 mg/ml fusionA pH 5.0 stored at 2-8° C.

SEC results for content fusionA monomer after 12 weeks of storage of 25 mg/ml fusionA liquid formulation pH 5.0 at 2-8° C. shown in FIG. 56.

SEC results for purity fusionA monomer after 12 weeks of storage of 25 mg/ml fusionA liquid formulation pH 5.0 at 2-8° C. shown in FIG. 57.

C2) Liquid samples of 25 mg/ml fusionA pH 7.0 stored at 2-8° C.

SEC results for content fusionA monomer after 12 weeks of storage of 25 mg/ml fusionA liquid formulation pH 7.0 at 2-8° C. shown in FIG. 58.

SEC results for purity fusionA monomer after 12 weeks of storage of 25 mg/ml fusionA liquid formulation pH 7.0 at 2-8° C. shown in FIG. 59.

The storage of the liquid fusionA formulations in the fridge at 2-8° C. does show only a slight reduction in content for both tested pH values. The monomer purities are at the same level for pH 5.0 for every formulation, whereas the pH 7.0 formulations show a slight reduction in the purity values for each tested solution. As each formulation is on the same level of content and purity it can be concluded that the meglumine salts are suitable to stabilize proteins as well as the prominent substances sucrose, trehalose and arginine-glutamate.

### LIST OF FIGURES

FIG. 1 Example 1 A) stabilizing effect of meglumine-glutamate and meglumine aspartate vs. meglumine and sucrose towards the melting temperature (Tm) of mAbA formulated at 1 mg/ml in McIlvaine-buffer pH 5

FIG. 2 Example 1 B) stabilizing effect of meglumine-glutamate and meglumine aspartate vs. meglumine and sucrose towards the melting temperature (Tm) of mAbB formulated at 1 mg/ml in McIlvaine-buffer pH 5

FIG. 3 Example 1 C) stabilizing effect of meglumine-glutamate and meglumine aspartate vs. meglumine and sucrose towards the melting temperature (Tm) of the fusion protein fusionA formulated at 1 mg/ml in McIlvaine-buffer pH 5

FIG. 4 Example 1 D) stabilizing effect of meglumine-glutamate and meglumine aspartate vs. meglumine and sucrose towards the onset temperature of aggregation (Tagg) for mAbA formulated at 1 mg/ml in McIlvaine-buffer pH 5

FIG. 5 Example 1 E): stabilizing effect of meglumine-glutamate and meglumine aspartate vs. meglumine and sucrose towards the onset temperature of aggregation (Tagg) for mAbB formulated at 1 mg/ml in McIlvaine-buffer pH 5

FIG. 6 Example 2 A) stabilizing effect of meglumine-glutamate (Meg-Glu), meglumine-lactobionate (Meg-Lac) and meglumine aspartate (Meg-Asp) vs. meglumine and sucrose towards the melting temperature (Tm) of mAbA formulated at 50 mg/ml in 10 mM citrate buffer pH 5

FIG. 7 Example 2 B) stabilizing effect of meglumine-glutamate (Meg-Glu), meglumine-lactobionate (Meg-Lac) and meglumine aspartate (Meg-Asp) vs. meglumine and sucrose towards the melting temperature (Tm) of mAbB formulated at 50 mg/ml in 10 mM citrate buffer pH 5

FIG. 8 Example 2 C) stabilizing effect of meglumine-glutamate (Meg-Glu), meglumine-lactobionate (Meg-Lac) and meglumine aspartate (Meg-Asp) vs. meglumine and sucrose towards the melting temperature (Tm) of fusionA formulated at 50 mg/ml in 10 mM citrate buffer pH 5

FIG. 9 Example 2 D) stabilizing effect of meglumine-glutamate (Meg-Glu), meglumine-lactobionate (Meg-Lac) and meglumine aspartate (Meg-Asp) vs. meglumine and sucrose towards the onset temperature of aggregation (Tagg) for mAbA formulated at 50 mg/ml in 10 mM citrate buffer pH 5

FIG. 10 Example 2 E) stabilizing effect of meglumine-glutamate (Meg-Glu), meglumine-lactobionate (Meg-Lac) and meglumine aspartate (Meg-Asp) vs. meglumine and sucrose towards the onset temperature of aggregation (Tagg) for mAbB formulated at 50 mg/ml in 10 mM citrate buffer pH 5

FIG. 11 Example 2 F) stabilizing effect of meglumine-glutamate (Meg-Glu), meglumine-lactobionate (Meg-Lac) and meglumine aspartate (Meg-Asp) vs. meglumine and sucrose towards the onset temperature of aggregation (Tagg) for fusionA formulated at 50 mg/ml in 10 mM citrate buffer pH 5

FIG. 12 Remaining protein-monomer concentration [mg/ml] of mAbA at a concentration of 1 mg/ml formulated in a phosphate/citrate buffer (McIlvaine buffer) after isothermal stress at 60° C. for 180 minutes with varying concentrations of an equimolar mixture of meglumine and glutamate.

FIG. 13 Remaining protein-monomer concentration [mg/ml] of mAbA at a concentration of 1 mg/ml formulated in a phosphate/citrate buffer (McIlvaine buffer) after isothermal stress at 60° C. for 180 minutes with varying concentrations of meglumine

FIG. 14 Remaining protein-monomer concentration [mg/ml] of mAbA at a concentration of 1 mg/ml formulated in a phosphate/citrate buffer (McIlvaine buffer) after isothermal stress at 60° C. for 180 minutes with varying concentrations of meglumine.

FIG. 15 Example 4A) Turbidity measurement at 350 nm after storage at 40° C. at 75% r.H for 0 weeks (initial value), 4 weeks, 8 weeks and 12 weeks.

FIG. 16 Example 4B) SEC measurement after storage at 40° C. at 75% r.H for 0 weeks (initial value), 4 weeks, 8 weeks and 12 weeks.

FIG. 17 Example 5A: Turbidity measurement at 350 nm after isothermal stress

FIG. 18 Example 5B: SEC measurement after isothermal stress

FIG. 19 Example 6A: Turbidity measurement at 350 nm during stability test at 2, 4, 9 and 12 weeks

FIG. 20 Example 6B: SEC analysis during stability test at 2, 4, 9 and 12 weeks

FIG. 21 Example 7: Tm values for mabB 50 mg/ml stabilized with 50 mM/250 mM meglumine at pH 5 and pH 7

FIG. 22 Example 7: Tagg values for mabB 50 mg/ml stabilized with 50 mM/250 mM meglumine at pH 5 and pH 7

FIG. 23 Example 7: Tm values for mabB 50 mg/ml stabilized with 50 mM/250 mM sucrose at pH 5 and pH 7

FIG. 24 Example 7: Tagg values for mabB 50 mg/ml stabilized with 50 mM/250 mM sucrose at pH 5 and pH 7

FIG. 25 Example 7: Tm values for mabB 50 mg/ml stabilized with 50 mM/250 mM meglumine-glutamate at pH 5 and pH 7

FIG. 26 Example 7: Tagg values for mabB 50 mg/ml stabilized with 50 mM/250 mM meglumine-glutamate at pH 5 and pH 7

FIG. 27: Sample volumes for preparation of fusionA samples for stability study at pH 5.0 FIG. 28: Sample volumes for preparation of fusionA samples for stability study at pH 7.0

FIG. 29: Conditions for freeze-drying

FIG. 30: Sub-visual particles >10 μm after 0, 4, 8 or 12 weeks of storage of 25 mg/ml fusionA liquid formulation pH 5.0 at 25° C./60% r. H.

FIG. 31: Sub-visual particles >25 μm after 0, 4, 8 or 12 weeks of storage of 25 mg/ml fusionA liquid formulation pH 5.0 at 25° C./60% r. H.

FIG. 32: Comparison of 50 mM and 200 mM sucrose and meglumine-glutamate in terms of sub-visual particles for 25 mg/ml fusionA liquid formulation pH 5.0 stored for 0, 4, 8 or 12 weeks at 25° C./60% r.H.

FIG. 33: Sub-visual particles >10 μm after 0, 4, 8 and 12 weeks of storage of 25 mg/ml fusionA liquid formulation pH 7.0 at 25° C./60% r.H.

FIG. 34: Sub-visual particles >25 μm after 0, 4, 8 and 12 weeks of storage of 25 mg/ml fusionA liquid formulation pH 7.0 at 25° C./60% r. H.

FIG. 35: Comparison of 50 mM and 200 mM sucrose and meglumine-glutamate in terms of sub-visual particles for 25 mg/ml fusionA liquid formulation pH 7.0 stored for 0, 4, 8 or 12 weeks at 25° C./60% r. H.

FIG. 36: Sub-visual particles >10 μm after 0, 4 and 12 weeks of storage of 25 mg/ml fusionA freeze-dried formulation pH 5.0 at 25° C./60% r.H.

FIG. 37: Sub-visual particles >25 μm after 0, 4 and 12 weeks of storage of 25 mg/ml fusionA freeze-dried formulation pH 5.0 at 25° C./60% r. H.

FIG. 38: Comparison of 50 mM and 200 mM sucrose and meglumine-glutamate in terms of sub-visual particles for 25 mg/ml fusionA freeze-dried formulation pH 5.0 stored for 0, 4 and 12 weeks at 25° C./60% r.H.

FIG. 39: Sub-visual particles >10 μm after 0, 4 and 12 weeks of storage of 25 mg/ml fusionA freeze-dried formulation pH 7.0 at 25° C./60% r. H.

FIG. 40: Sub-visual particles >25 μm after 0, 4 and 12 weeks of storage of 25 mg/ml fusionA freeze-dried formulation pH 7.0 at 25° C./60% r. H.

FIG. 41: Comparison of 50 mM and 200 mM sucrose and meglumine-glutamate in terms of sub-visual particles for 25 mg/ml fusionA freeze-dried formulation pH 7.0 stored for 0, 4 and 12 weeks at 25° C./60% r. H.

FIG. 42: Sub-visual particles >10 μm after 12 weeks of storage of 25 mg/ml fusionA freeze-dried formulation pH 5.0 at 2-8° C.

FIG. 43: Sub-visual particles >25 μm after 12 weeks of storage of 25 mg/ml fusionA freeze-dried formulation pH 5.0 at 2-8° C.

FIG. 44: Comparison of 50 mM and 200 mM sucrose and meglumine-glutamate in terms of sub-visual particles for 25 mg/ml fusionA liquid formulation pH 5.0 stored at 2-8° C.

FIG. 45: Sub-visual particles >10 μm after 12 weeks of storage of 25 mg/ml fusionA freeze-dried formulation pH 7.0 at 2-8° C.

FIG. 46: Sub-visual particles >25 μm after 12 weeks of storage of 25 mg/ml fusionA freeze-dried formulation pH 7.0 at 2-8° C.

FIG. 47: Comparison of 50 mM and 200 mM sucrose and meglumine-glutamate in terms of sub-visual particles for 25 mg/ml fusionA liquid formulation pH 7.0 stored at 2-8° C.

FIG. 48: SEC results for content fusionA monomer after 0, 4, 8 and 12 weeks of storage of 25 mg/ml fusionA liquid formulation pH 5.0 at 25° C./60% r. H.

FIG. 49: SEC results for purity fusionA monomer after 0, 4, 8 and 12 weeks of storage of 25 mg/ml fusionA liquid formulation pH 5.0 at 25° C./60% r. H.

FIG. 50: SEC results for content fusionA monomer after 0, 4, 8 and 12 weeks of storage of 25 mg/ml fusionA liquid formulation pH 7.0 at 25° C./60% r. H.

FIG. 51: SEC results for purity fusionA monomer after 0, 4, 8 and 12 weeks of storage of 25 mg/ml fusionA liquid formulation pH 7.0 at 25° C./60% r. H.

FIG. 52: SEC results for content fusionA monomer after 0, 4 and 12 weeks of storage of 25 mg/ml fusionA freeze-dried formulation pH 5.0 at 25° C./60% r. H.

FIG. 53: SEC results for purity fusionA monomer after 0, 4 and 12 weeks of storage of 25 mg/ml fusionA freeze-dried formulation pH 5.0 at 25° C./60% r.H.

FIG. 54: SEC results for content fusionA monomer after 0, 4 and 12 weeks of storage of 25 mg/ml fusionA freeze-dried formulation pH 7.0 at 25° C./60% r. H.

FIG. 55: SEC results for purity fusionA monomer after 0, 4 and 12 weeks of storage of 25 mg/ml fusionA freeze-dried formulation pH 7.0 at 25° C./60% r. H.

FIG. 56: SEC results for content fusionA monomer after 12 weeks of storage of 25 mg/ml fusionA liquid formulation pH 5.0 at 2-8° C.

FIG. 57: SEC results for purity fusionA monomer after 12 weeks of storage of 25 mg/ml fusionA liquid formulation pH 5.0 at 2-8° C.

FIG. 58: SEC results for content fusionA monomer after 12 weeks of storage of 25 mg/ml fusionA liquid formulation pH 7.0 at 2-8° C.

FIG. 59: SEC results for purity fusionA monomer after 12 weeks of storage of 25 mg/ml fusionA liquid formulation pH 7.0 at 2-8° C.

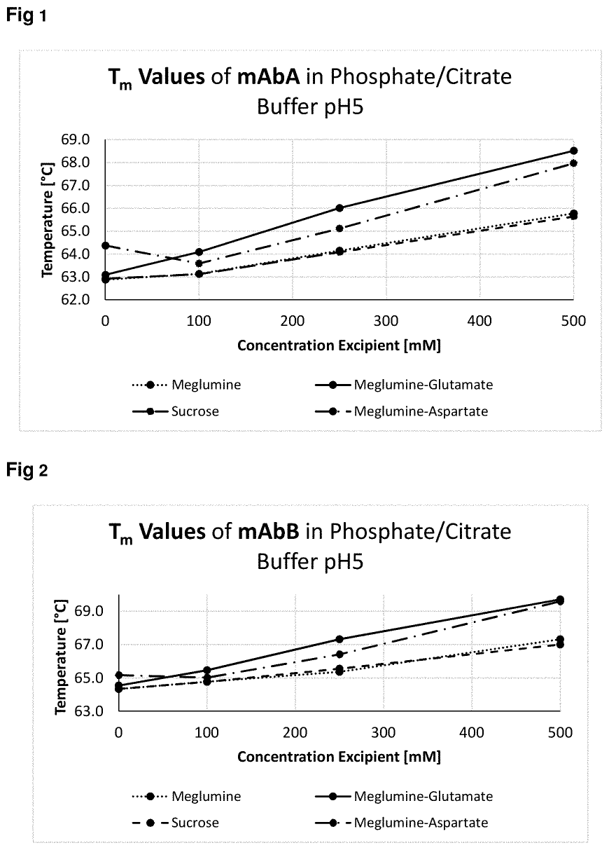

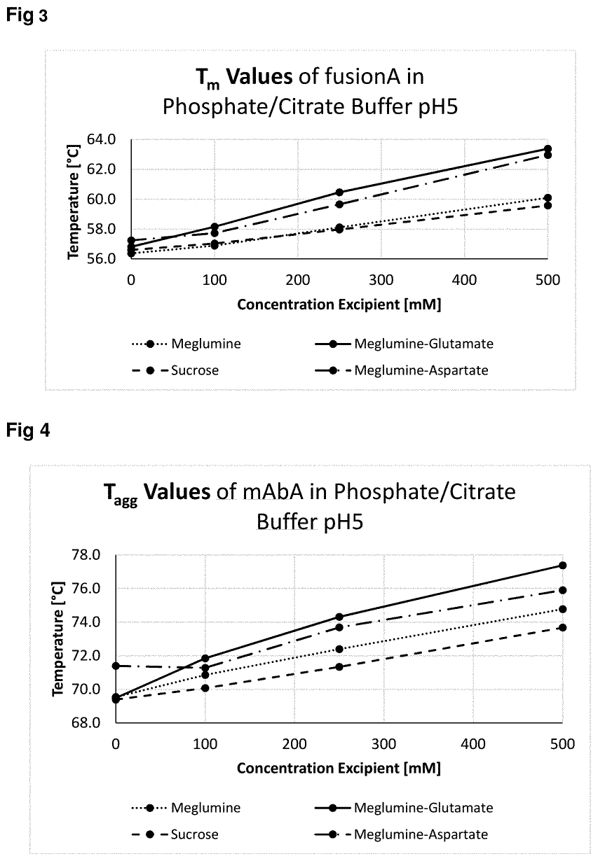

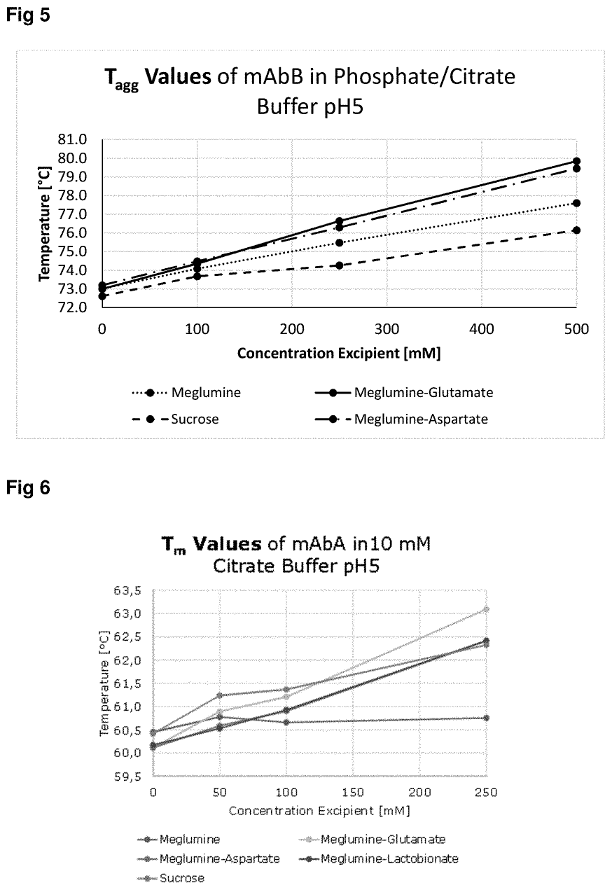

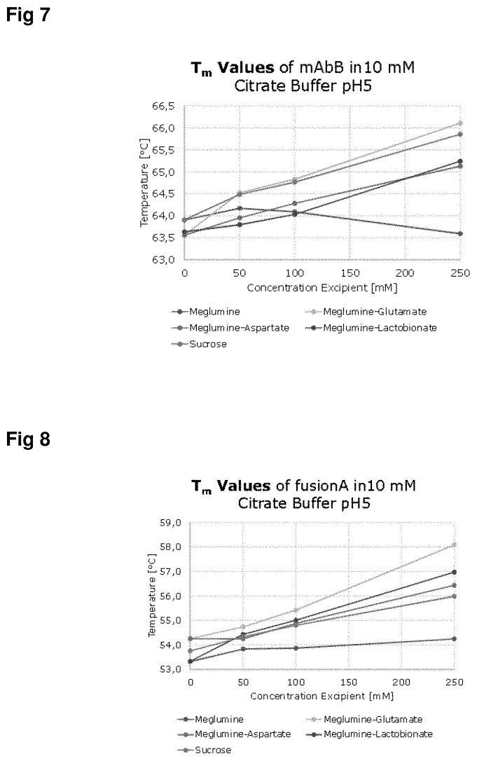

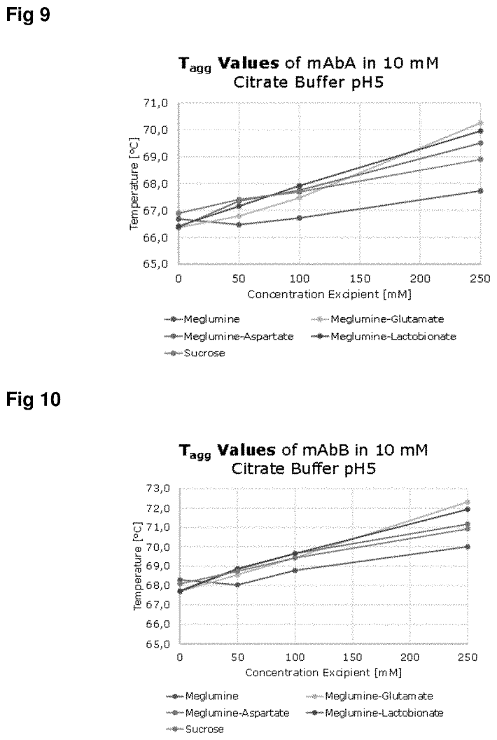

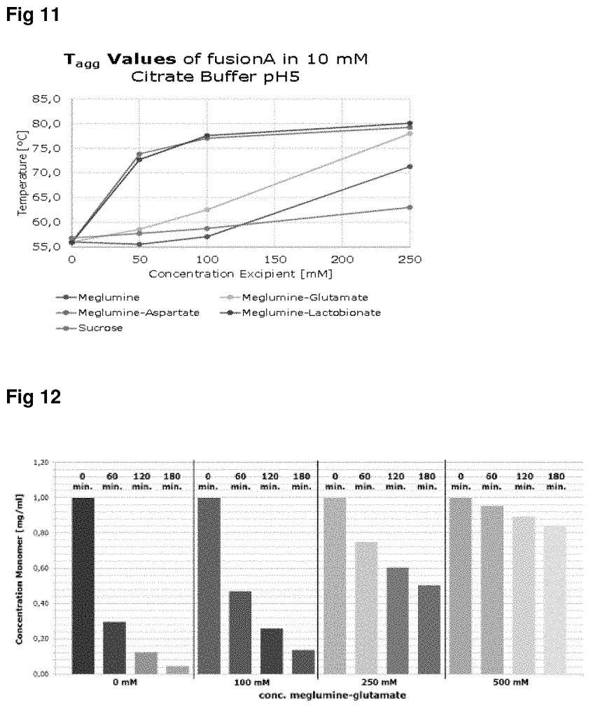

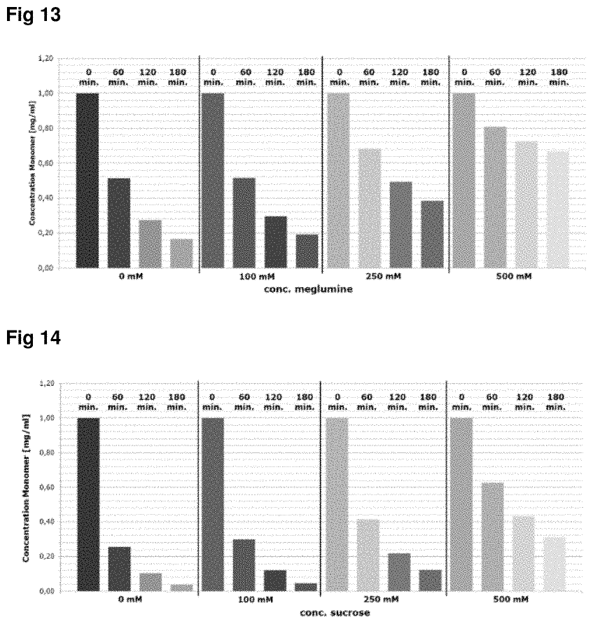

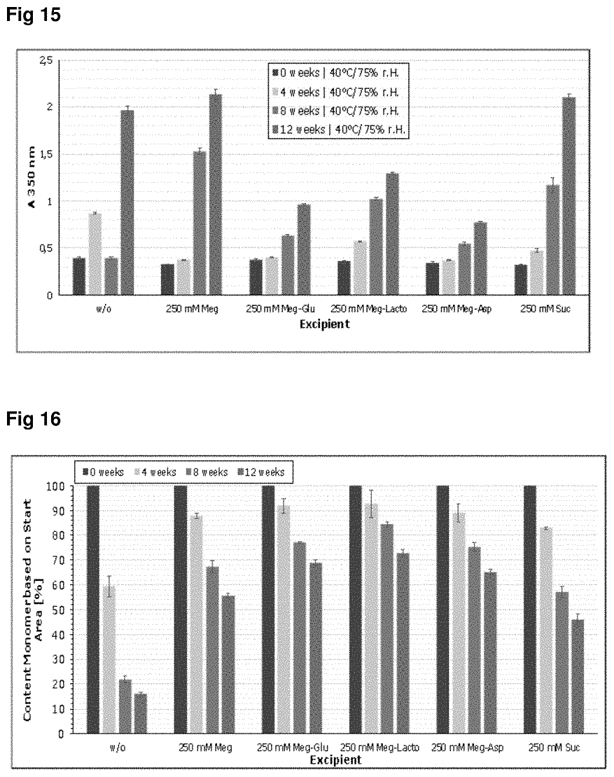

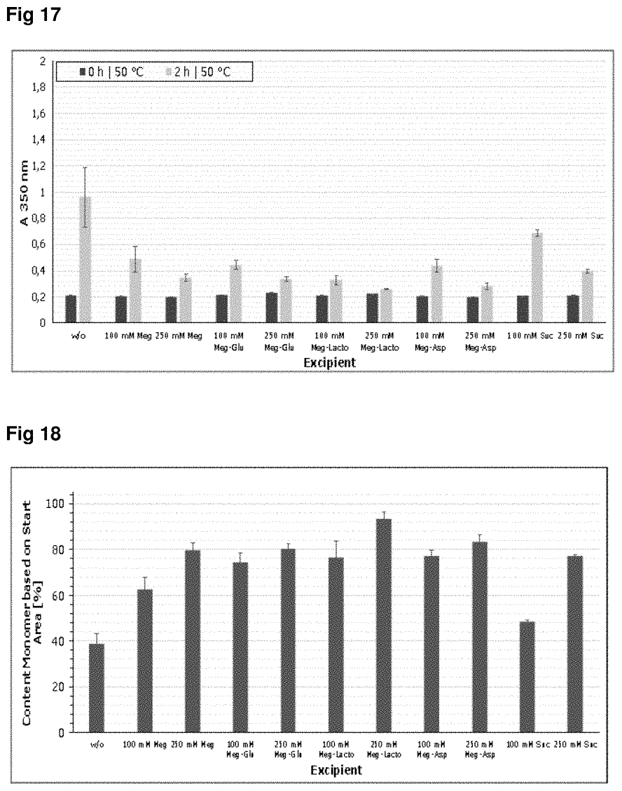

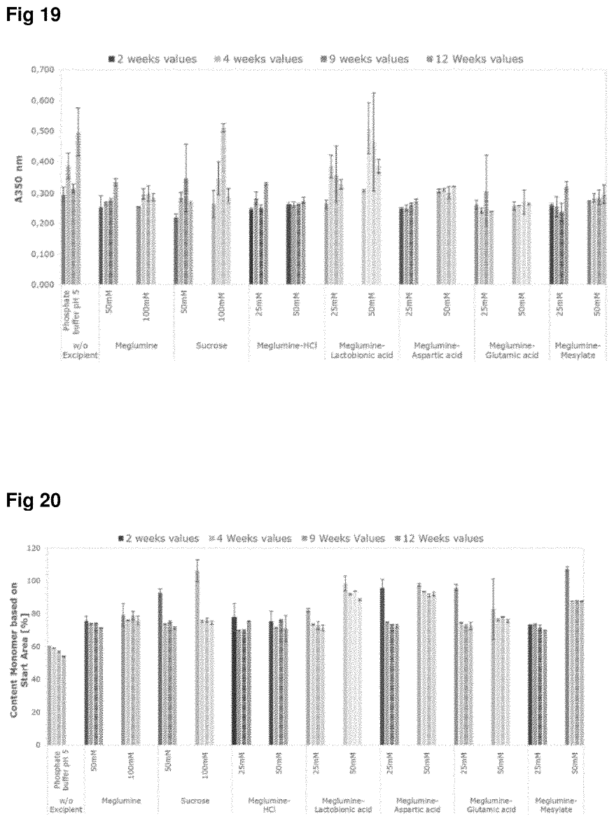

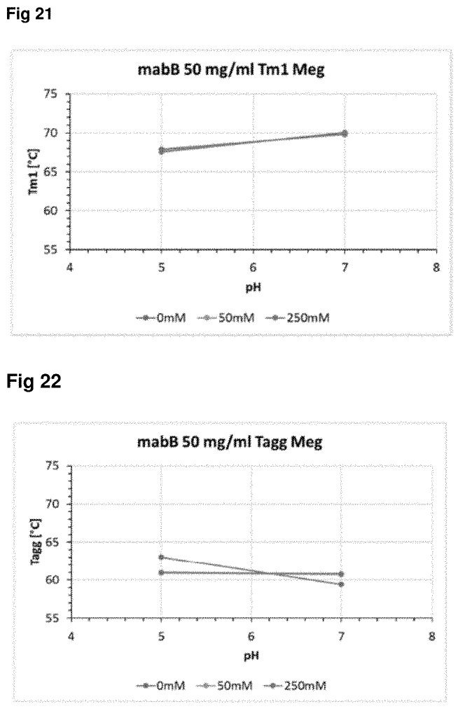

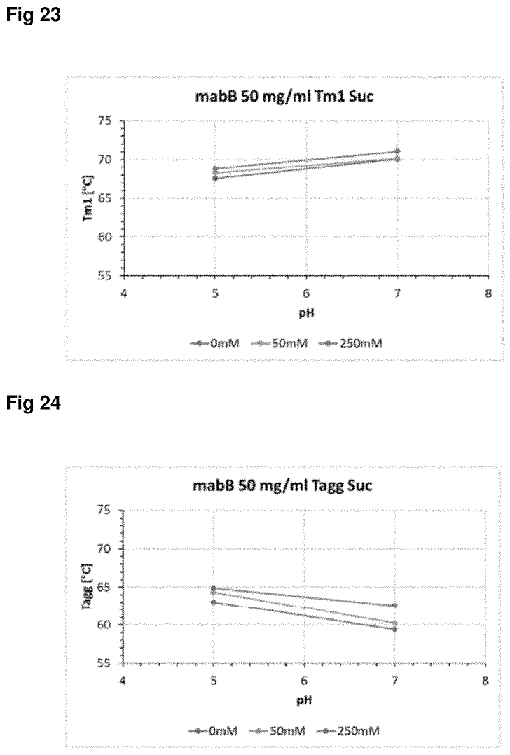

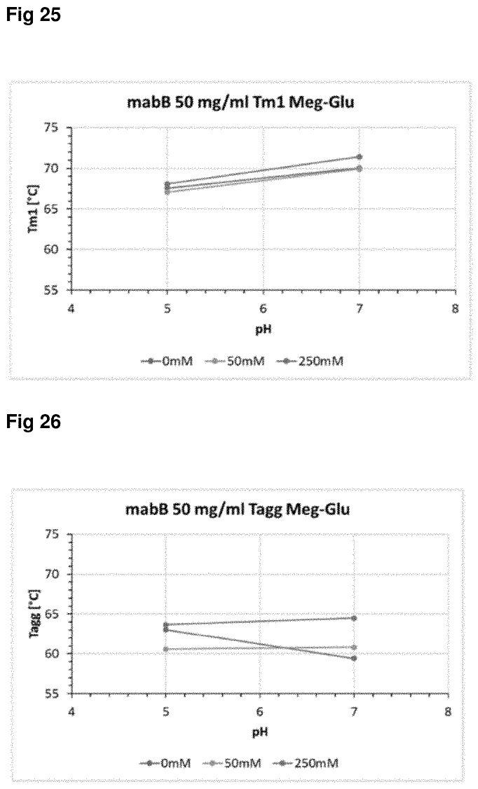

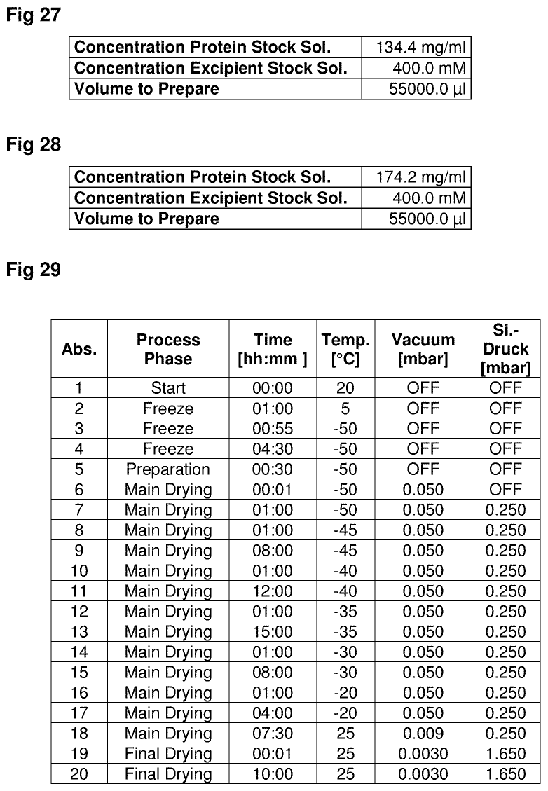

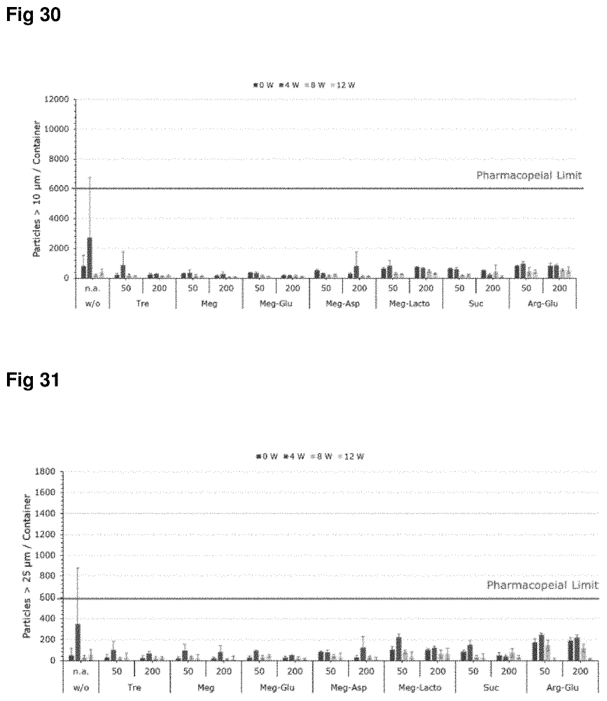

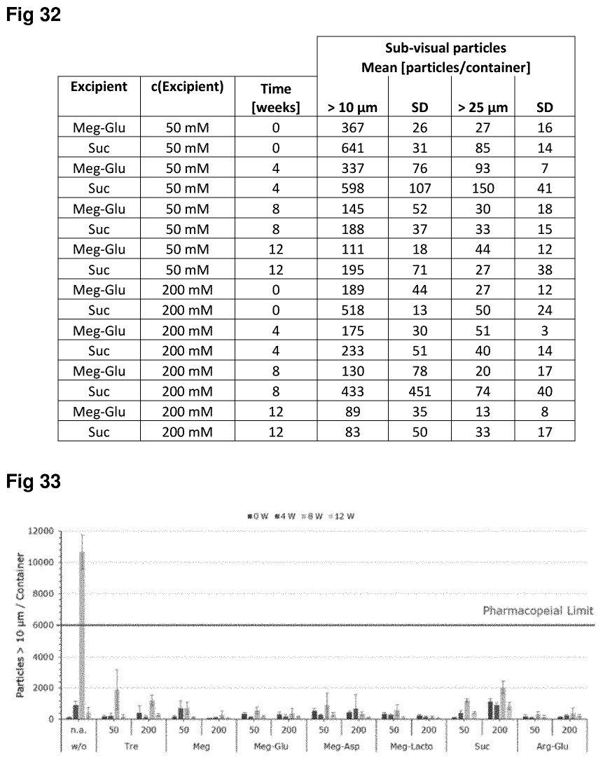

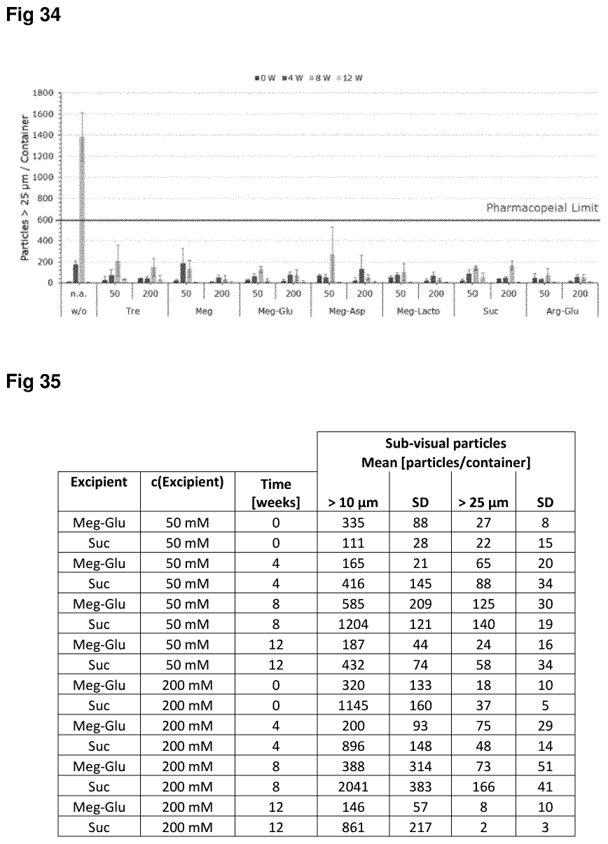

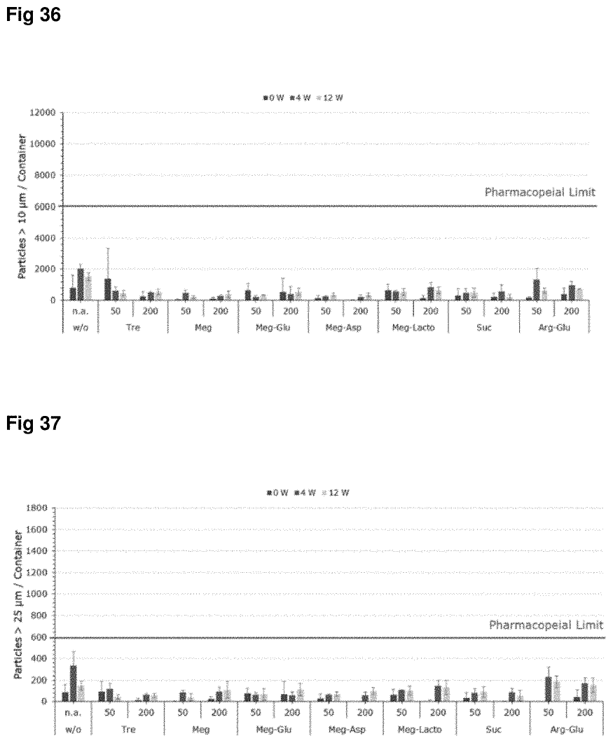

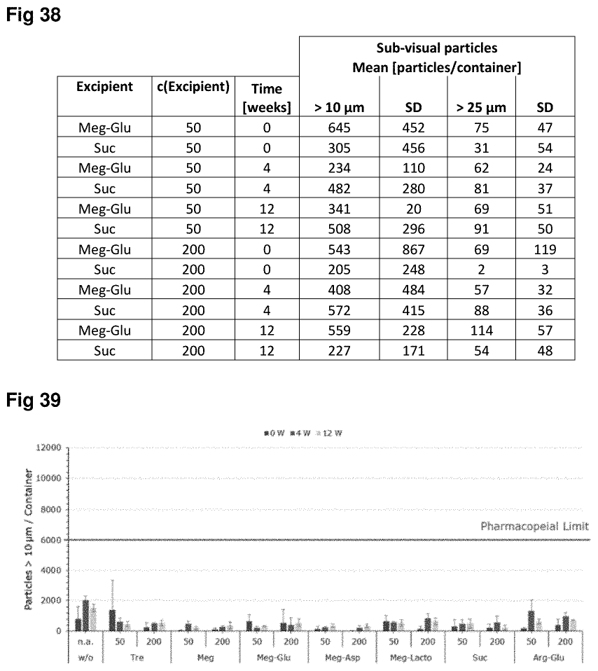

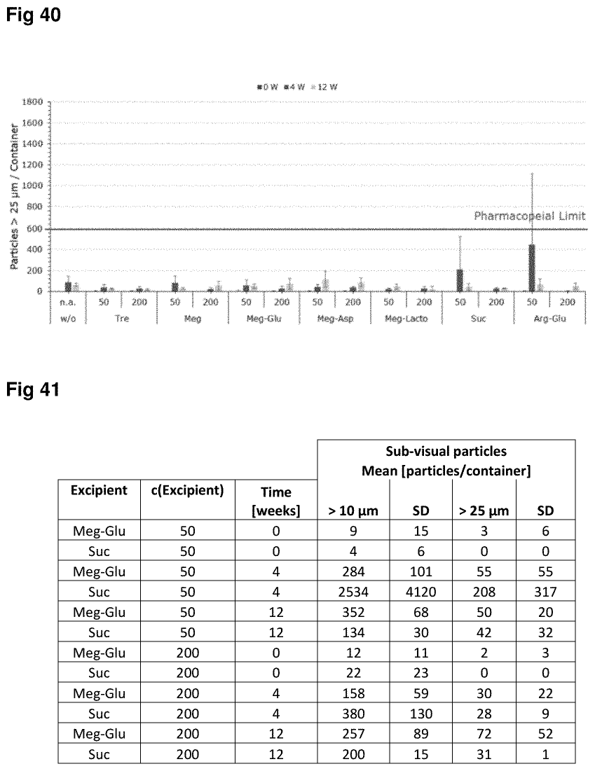

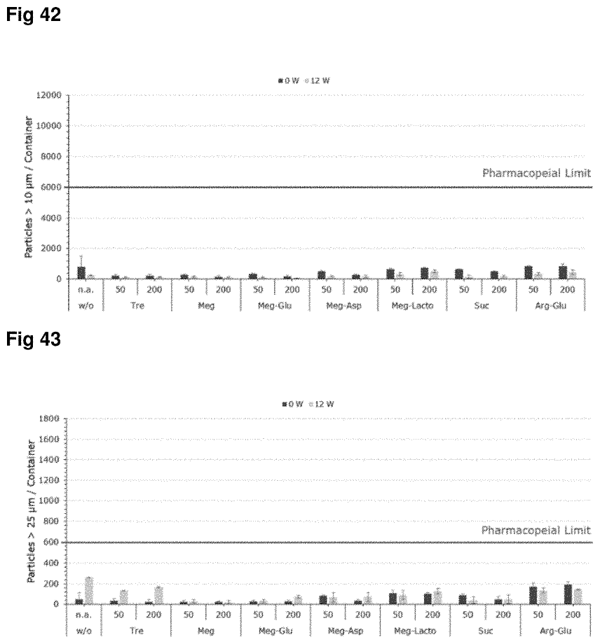

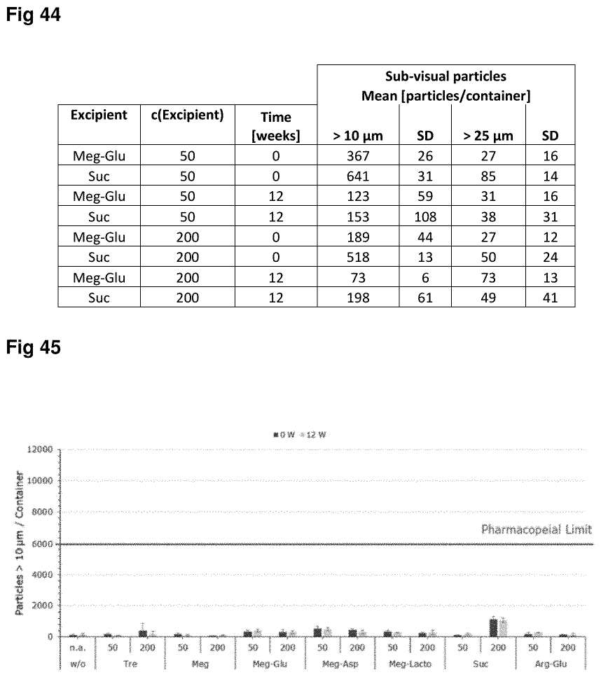

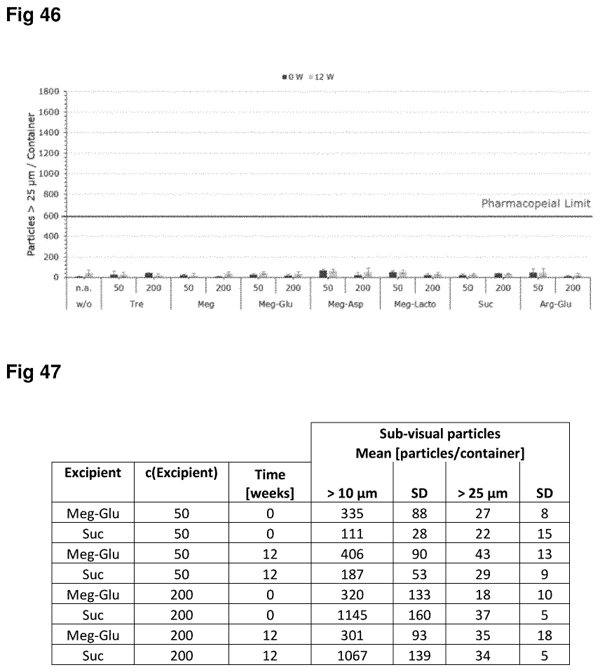

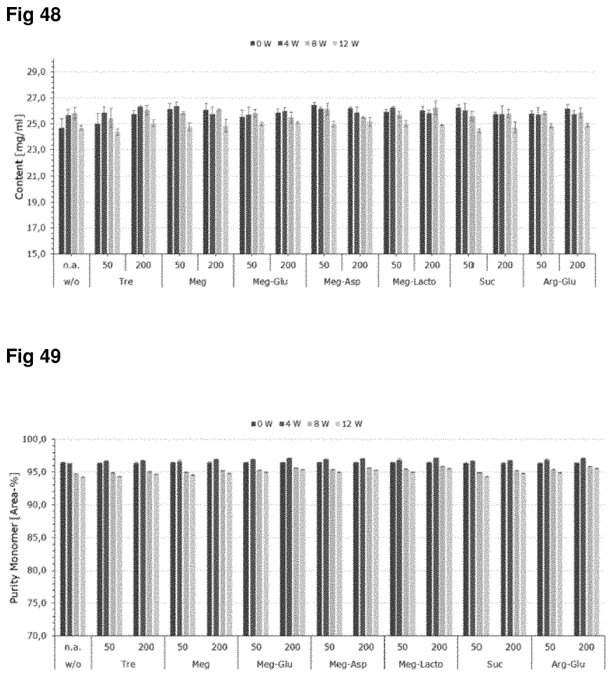

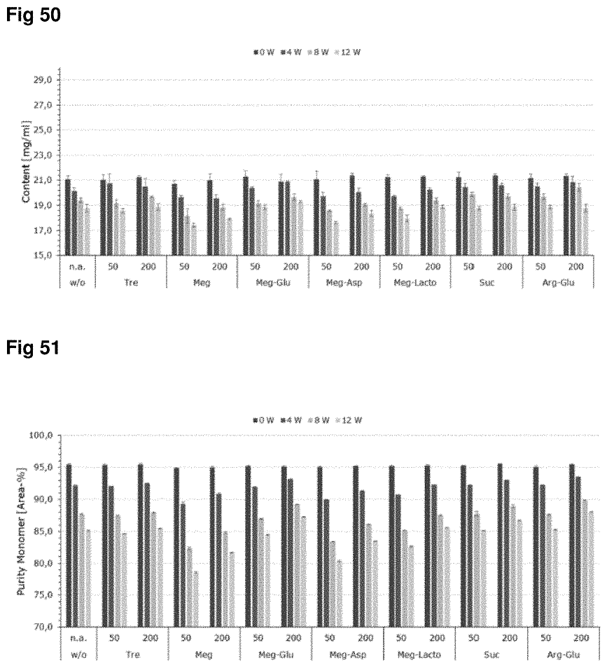

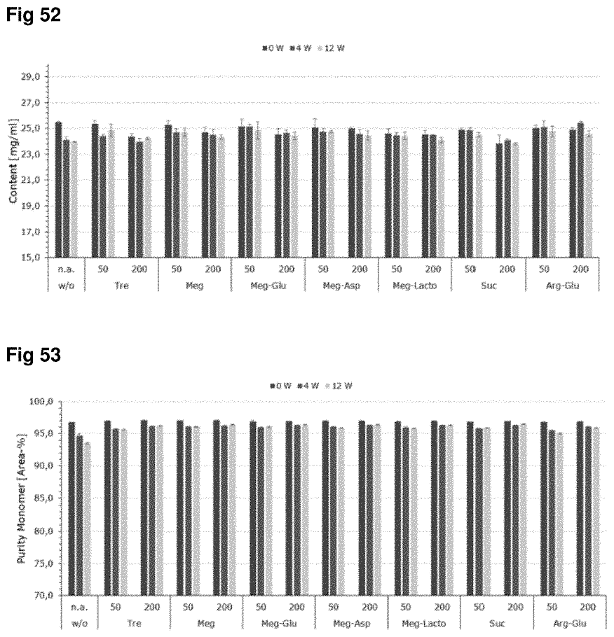

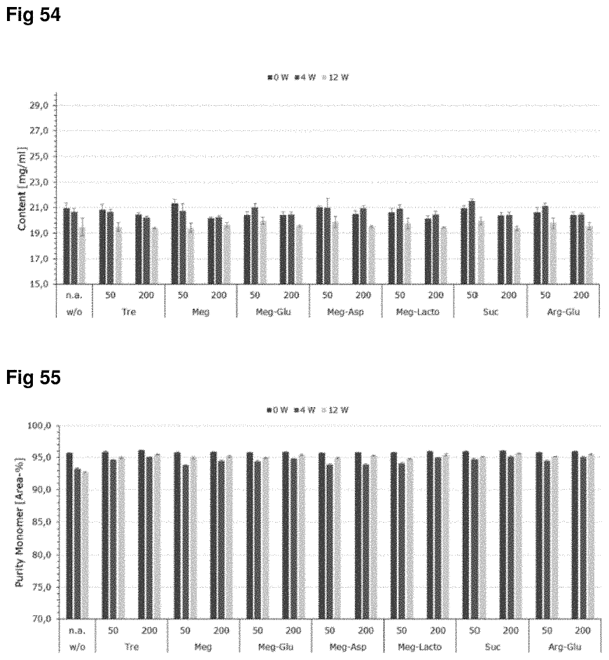

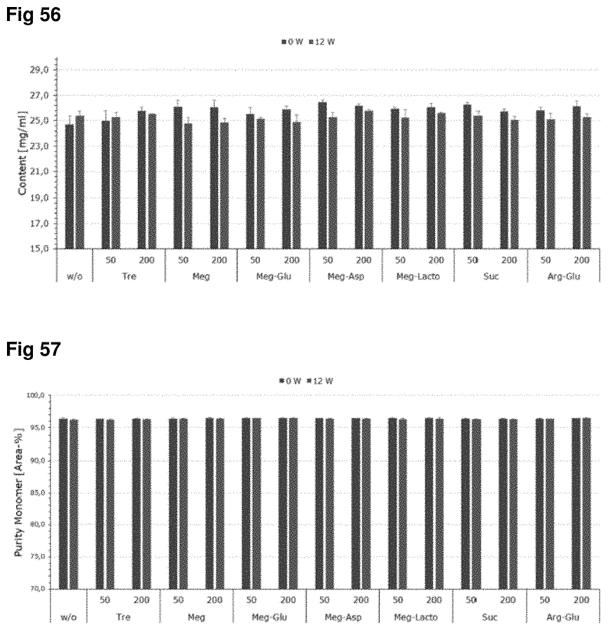

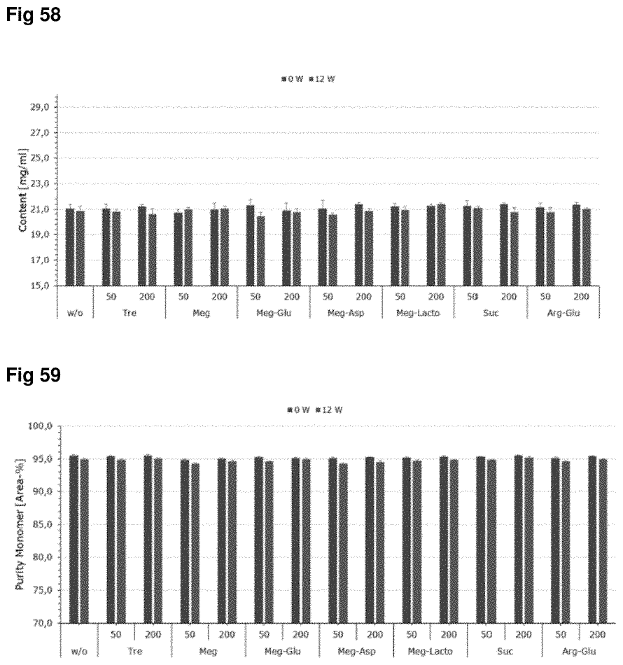

1. A method of stabilizing of a liquid protein or peptide formulation or for suppressing protein aggregation in said formulation by treatment of the peptide- or protein-containing solution with a combination of meglumine and a physiologically well-tolerated organic counterion in effective concentrations to stabilize the protein or peptide molecules contained therein.

2. Method of claim 1 for the stabilization of a liquid protein or peptide formulation or for suppressing protein aggregation by
(a) providing a first solution comprising protein or peptide molecules; and
(b) providing a second solution comprising meglumine in combination with a physiologically well-tolerated organic counterion in a suitable formulation,
(c) adding a sufficient amount of the second solution to the first solution,
thereby setting in the resulting mixture a meglumine-counterion concentration, which is effective for stabilization the comprising protein or peptide molecules.

3. Method according to claim 1, wherein the counterion is selected either from the group of compounds having at least one carboxylic acid group and at least one amino group, but no aromatic groups in the molecule,
or selected from the group of compounds having at least one carboxylic acid group, at least one amino group and at least one OH group, but no aromatic groups in the molecule,
or selected from the group of compounds having at least one carboxylic acid group and at least two or more OH groups, but no aromatic groups in the molecule.

4. Method according to claim 1, wherein the counterion is selected from the group of glutamate, aspartate, lactate and lactobionate.

5. Method according to claim 1, wherein the protein is selected from the group of antibodies, antibody fragments, minibody, modified antibody, antibody-like molecules and fusion proteins.

6. Method according to claim 1, wherein the protein molecules are antibody molecules.

7. Method of claim 1, wherein the liquid protein or peptide formulation is a pharmaceutical composition.

8. Method of claim 1, wherein after addition of the meglumine in combination with a counterion to the first solution the pH is adjusted within the range of pH 5 to 8.

9. Method of claim 1, wherein after addition of the meglumine in combination with a counterion to the first solution the pH is adjusted within a range of 7.2 to 7.6, preferably at pH 7.4.

10. Method of claim 1, setting in the resulting mixture a molar ratio of meglumine to counterion in the range of 1:1 up to 1:2, which is effective for stabilization the comprising protein or peptide molecules.

11. Method of claim 1, setting in the resulting mixture a molar ratio of meglumine to counterion of 1:1, which is effective for stabilization the comprising protein or peptide molecules.

12. Method according to claim 1, whereby protein or peptide solutions with protein or peptide concentrations in the range between 1 mg/ml up to 500 mg/ml are stabilized.

13. Method according to claim 1, wherein for stabilization of proteins or peptides the meglumine concentration is adjusted at a high concentration in the range of 1 mM to 1.5 M in the solution.

14. Method according to claim 1, wherein for stabilization of proteins or peptides the meglumine concentration is adjusted at a concentration in the range of 5 mM to 500 mM in the solution.

15. Method according to claim 1, whereby proteins or peptides are stabilized and denaturation and aggregation are suppressed under long-term storage conditions at room temperature.

16. Method according to claim 1, whereby proteins or peptides are stabilized and denaturation and aggregation are suppressed under long-term storage conditions for three months at 40° C. and relative humidity of 75% rel.

17. Method according to claim 1, whereby proteins or peptides are stabilized and denaturation and aggregation are suppressed under long-term storage conditions at low temperatures in the range of −80° C. to 10° C.

18. Method according to claim 1, further freeze-drying the resulting mixture after the step of adding the solution comprising meglumine in combination with a counterion, to produce a freeze-dried preparation.

19. A pharmaceutical composition, obtainable by a method according to claim 1, comprising an antibody molecule and a meglumine salt, selected from the group of meglumine glutamate, meglumine aspartate and meglumine lactobionate.

20. Pharmaceutical composition of claim 18, whose dosage form is a freeze-dried preparation.

21. Kit comprising a pharmaceutical composition, obtainable by a method according to claim 1, and pharmaceutically acceptable carrier.

22. Kit according to claim 21, comprising freeze-dried or spray-dried preparations of a pharmaceutical composition, which can be made into solution preparations prior to use.

23. Kit according to claim 21, comprising ready-to-use freeze-dried or spray-dried formulations sitting in a 96-well plate.

24. Kit according to claim 21 for administration to patients, including a container, syringe and/or other administration device with or without needles, infusion pumps, jet injectors, pen devices, transdermal injectors, or other needle-free injector and instructions.
